(19) United States
(12) Patent Application Publication
(10) Pub. No.: US 2007/0135365 A1
(43) Pub. Date: Jun. 14, 2007

(54) Pharmaceutical composition for preventing or remedying cardiac hypertrophy and cardiovascular disease caused thereby

(76) Inventors: Katsuyuki Tanizawa, Toyono-gun (JP); Miki Iwata, Yawata-shi (JP); Shun'ichi Kuroda, Suita-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/568,915

(22) PCT Filed: Aug. 20, 2004
(86) PCT No.: PCT/JP04/12336

§ 371 Date: Aug. 28, 2006

Related U.S. Application Data
(60) Provisional application No. 60/496,935, filed on Aug. 21, 2003.

(51) Int. Cl. A61K 48/00

(52) U.S. Cl. 514/44

(57) ABSTRACT

Compositions and methods for preventing or treating cardiac hypertrophy which inhibit the cardiac hypertrophy signal pathway mediated through G protein coupled receptors (GPCR) or epidermal growth factor (EGF) receptors. The present invention also relates to methods for screening active ingredients, and to animal models of cardiac hypertrophy.

### TECHNICAL FIELD

The present invention offers a new understanding that relates to the mechanisms inducing cardiac hypertrophy, and more particularly to the signal transmission route leading to cardiac hypertrophy. The present invention also relates to a composition,to suppress the onset of cardiac hypertrophy based on the findings in question (composition to suppress cardiac hypertrophy). The present invention further relates to a composition that, based on the above action, is used to prevent or remedy the onset of impaired cardiac function such as heart failure specifically caused by cardiac hypertrophy (composition to prevent or remedy heart disease caused by cardiac hypertrophy).

Furthermore, the present invention relates to a method to suppress cardiac hypertrophy in patients based on the new understanding related to the mechanism generating cardiac hypertrophy, and relates to a method to prevent or remedy heart disease caused by cardiac hypertrophy (specifically, impaired cardiac function such as heart failure).

The present invention also relates to a method to screen the active ingredients of the aforementioned composition (composition to suppress cardiac hypertrophy, or composition to prevent or remedy heart disease caused by cardiac hypertrophy). The present invention further relates to a disease animal model to show the disease of cardiac hypertrophy.

### BACKGROUND ART

The heart is an organ that is differentiated extremely early during genesis of the individual, and begins to beat autonomously immediately after differentiation. Cardiomyocytes maintain a capacity to divide even after differentiation, and continue to actively increase by division in the fetal period, but that capacity for growth suddenly drops after birth. As a consequence, it appears that post-natal cardiomyocytes have no capacity for regeneration, and that subsequent growth of the heart occurs only by physiological enlargement, specifically, by increasing the size of the individual cardiomyocytes. Enlargement of the heart (cardiac hypertrophy) is caused either by an increase of the width of the myoblast fibers (this produces thickening of the heart walls, specifically, “concentric hypertrophy”), or by an increase of the length of the myoblast fibers (this produces expansion of the chambers, specifically, “eccentric hypertrophy”). These contrasting hypertrophic forms are derived respectively by parallel assembly and serial assembly of the sarcomeres.

Cardiac hypertrophy is induced by response to post-natal physiological adaptation or by movement, but this is a normal physiological phenomenon because a balance is simultaneously produced between the aforementioned concentric hypertrophy and eccentric hypertrophy, the pump transport capacity of the heart is increased corresponding to the increase in the amount of demand.

Meanwhile, a pathologically generated load on the heart may also induce cardiac hypertrophy. Specifically, when the load on the ventricles is increased by hypertension or valvular disease of the heart, or when damage to the cardiomyocytes themselves is produced by myocardial infarction, myocarditis or myocardosis, cardiac hypertrophy occurs, specifically, the heart changes shape, mainly by hypertrophic growth of cardiomyocytes, in order to maintain cardiac output. Up to a certain extent, this kind of cardiac hypertrophy appears to be a compensatory phenomenon for impairment of cardiomyocytes and mechanical load, but if the excess load on the heart is applied continually and notable hypertrophy occurs, the systolic and diastolic functions of the heart breakdown, chronic heart failure appears based on decreased cardiac output, and the heart becomes susceptible to ischemic heart disease and prone to fatal arrhythmia. In this type of pathological load on the heart one or the other of concentric hypertrophy or eccentric hypertrophy may predominate, and even if not leading to heart failure, hypertrophic myocardosis or eccentric myocardosis may occur.

Cardiac hypertrophy has lately become recognized as one of the independent risk factors leading to coronary disease such as heart failure, and the Framingham Heart Study, which was a large-scale follow-up study conducted in the US, demonstrated that when cardiac hypertrophy is present, there is a 2.5 to 3 fold increase in the percentage of onset of heart failure, ischemic heart diseases such as angina pectoris and myocardial infarction, and cardiovascular diseases such as arrhythmia (Chikara Yamazaki, Yoshio Yazaki, “Cardiac Failure”:, pages 37-45, Shigetake Shinoyama ed., Iyaku Janarusha (Medicine & Drug Journal Co., Ltd.), 1997). Consequently, it appears that drugs to suppress excess formation of cardiac hypertrophy, or to cause regression of cardiac hypertrophy could be effectively used to prevent development of heart disease including chronic cardiac failure.

Past methods to treat chronic heart failure mainly use inotropic drugs with the objective of improving systolic capacity of the heart, and to increase the cardiac output. Nonetheless, although inotropic drugs indicate effects to acutely improve subjective symptoms and to improve exercise tolerance, there is no effect to improve the life prognosis, specifically, to prolong life, which is the ultimate goal of treating chronic cardiac failure. To the contrary, the results are that the prognosis worsens (Pacher et al., N. Engl. J. Med., Vol 325, p. 1468, 1991).

Meanwhile, the presence of cardiac hypertrophy signaling pathways relating to the mechanisms producing cardiac hypertrophy have been indicated. And, it is reported that the onset or development of cardiac hypertrophy is based on the activation of the signaling pathways by stimulus factors, subsequent protein synthesis, assembly and organization of sarcomeres, and regulation of gene expression (Chien, K. R., Cell, 98, p555-558, 1999; Nicol, R. L., et al., Ann. Rev. Gen. Gen., 1, p. 179-223, 2000; Sugden, P. H. et al., J. Mol. Med., 76, p. 725-746, 1998).

Well-known examples of the aforementioned stimulus factors are protein kinase (for example, the mitogen-activated protein kinase (MAPK) family such as ERK, JNK, and p38MAPK), and fluid factors (for example, vascular action substances such as angiotensin II, and endothelin-1, neural factors such as norepinephrine, cytokines such as cardiotrophin 1, and leukemia inhibitory factor (LIF), and growth factors such as cytokine, insulin, and IGF-1). In addition to being activated by a mechanical load such as extension of the cardiomyocytes, the aforementioned protein kinase is activated by fluid factors, and it appears that through the activation response transcription factors such as c-fos, c-myc, c-jun are activated, thus inducing proteins related to cardiac hypertrophy. Because mechanical stimulus and stimulus by angiotensin II, endothelin-1 and norepinephrine elevate intercellular calcium levels, and because cardiac hypertrophy is induced in mice that express constitutively active calcineurin (Olson et al., Cell, 93, p. 215-223, 1998), recent attention has focused on the role of calcium in the formation of cardiac hypertrophy. Moreover, because notable cardiac hypertrophy in the hearts of mice transiently expressed calmodulin was observed when the Ca²⁺/calmodulin-dependent protein kinase II (CaM kinase II) activity was elevated approximately 2 times (Mol Endocrinol 14, p. 1125-1136, 2000), and because cardiac hypertrophy was observed in mice with heart specific expression of constitutively active CaM kinase IV (J Clin Invest 105, p. 1395-1406, 2000), it appears that CaM kinase II and IV are also factors that stimulate formation of cardiac hypertrophy.

Therefore, with this object of suppressing the onset of cardiac hypertrophy is being made in the development and clinical application of drugs that inhibit the production of these stimulus factors or that suppress or block cardiac hypertrophy signaling through these factors (for example, angiotensin II production inhibitor, α1-blocker, endothelin receptor antagonist, etc.: J. Cardiovasc. Pharmacol., 27, S36-S40, 1996; Br. J. Pharmacol., 118, p. 549-556, 1996; Cardiovasc. Res., 23, p. 315-333, 1989; Circ. Res., 73, p. 887-897, 1993). However, it has been indicated that these factors have multiple relationships with the mechanisms of the onset of cardiac hypertrophy within the body, and an antagonistic action in relation to a single factor is insufficient. For example, although an inhibitor of angiotensin conversion enzyme (ACE), which is an enzyme that produces angiotensin II, suppresses the onset and development of cardiac hypertrophy in animal model s (Brilla et al., Circulation, Vol. 83, p. 1771, 1991), and causes regression of cardiac hypertrophy and extension of life prognosis when clinically administering to chronic heart failure patients (The save Investigation, N. Engl. J. Med., Vol. 327, p. 678, 1992), it could not yet be reported that the effects are sufficient. Actually, even with the most advanced therapies, the mortality five years after onset of chronic heart disease comes up to currently approximately 50%. There have been recent reports of endothelin antagonists (Ito et al., Circulation, Vol. 89, p. 2198, 1994) and vasopressin antagonists (Tumura et al., Circulation, Vol 94, (Supple. I-264), 1996) suppressing the formation of cardiac hypertrophy in animal models, but it is preferable to develop heart disease preventatives and remedies that can suppress cardiac hypertrophy based on even newer mechanisms.

Meanwhile, protein kinase D1 (called PKD1 hereinafter) is a protein comprising approximately 110 KDa (910-920 amino acid residues) that has a control region in the amino terminal region, and a catalytic region that codes protein kinase specific to serine-threonine in the carboxy terminal region. Further, in the aforementioned control region, there are a transmembrane region (TM), two CR (Cys-rich) domains comprising a continuous zinc finger (Cys-rich, Zn finger-like), and a PH (Pleckstrin Homology) domain (refer to FIG. 1).

The molecule of human-derived PKD1 is folded by the interaction between the CR domain of the control region and the catalytic region, and in this state is thought to be inactive (inactive PKD1). However, when phosphoinositide-dependent kinase 1: PDK1) acts, the PH domain or the active loop residue (Ser-744 [within PH], Ser-748 [a little downstream from PH]) positioned adjacent thereto is phosphorylated, and catalytically moves into the active state. Further, it has been demonstrated that the CR domains have a high affinity to Ca²⁺, diacylglycerol (DG) or phorbol ester (for example, TPA, etc.), and when these components are bound to the CR domains, the catalytic region separates from the CR domains, becomes all the more active, and then the molecule as a whole is fully activated by the catalytic region (Ser-916) of the C terminal undergoing self-phosphorylation (fully active PKD1) (Van Lint, J., et al., J. Biol. Chem. 3, p. 1455-1461, 1995; Zugaza, J. L., et al., EMBO J. 15, p. 6220-6230, 1996; Zugaza, J. L., et al., J. Biol. Chem. 272, p. 23952-23960, 1997; Iglesias, T., et al., J. Biol. Chem, 273, p. 27662-27667, 1998).

In the initial assay, PKD1 was identified as one (PKCμ) of the protein kinase C (called PKC hereinafter) family from the structural characteristics thereof, but for reasons such as the amino acid sequence of the catalytic region differs from that of the PKC family (the amino acid sequence of this region is highly conserved among the PKC family), it now appears that PKD1 belongs to an independent protein kinase family that differs from PKC. It has been indicated that several PKC isoforms (PKCη, PKCε, PKCβI) activate PKD1 based on phosphorylation of the active loop residue (Ser-744) of human-derived PKD1 and the residue adjacent thereto (Ser-748) (Maeda, Y., et al., EMBO J. 20, p. 5982-90, 2001; Waldron, R. T., et al., J. Biol. Chem. 274, p. 9224-9230, 1999; Waldron, R. T., et al., J. Biol. Chem. 276, p. 32606-32615, 2001).

PKD1 is expressed and is present in many tissues in the human body such as the brain, lungs, heart and skeletal muscles, and especially in the heart. It is known that some PKD1 is localized in the Golgi apparatus (Iglesias, T., et al., FEBS Lett. 454, p. 53-56, 1999; Jamora, C., et al., Cell. 98, p. 59-68, 1999; Liljedahl, M., et al., Cell. 104, p. 409-420, 2001), and plays an important role in Golgi function (Van Lint, J., et al., Trends in Cell Biol. 12, p. 193-200, 2002).

Thus, in the past the physiological function and role of PKD1 in the heart was completely unknown.

Moreover, it has been suggested regarding protein kinase C (PKC) that PKCε, which is one of the PKC isoforms, is notably expressed in the heart muscle, is localized in sarcomere Z-discs and is involved with cardiac hypertrophy (Takeishi, Y., et al., Circ. Res. 86, p. 1218-1223, 2000). Further, it has been reported that cardiac hypertrophy is generated when forcing expression of constitutively active PKCE using genetic recombination (Mochly-Rosen, D., et al., Circ Res., 86, p. 1173-1179, 2000).

Nonetheless, nothing at all was known either about the relationship of PKD1 and the onset and development of cardiac hypertrophy, or about the interaction between PKD1 and PKCε.

### BRIEF DESCRIPTION OF THE DRAWINGS

FIG. 1 is a conceptual diagram indicating the domain structure of protein kinase D1, and the structural changes of the inactive and active forms.

FIG. 2 indicates the Western blot results when comparing TPA (12-0-tetradecanolyphorbol 13-acetate)-treated and TPA-untreated (None) neonate rat cardiomyocytes (called NRC hereinafter) regarding the intracellular distributions (cytoplasm, membrane) of fully active PKD1 (phosphorylated PKD1) and inactive PKD1 (non-phosphorylated PKD1). TPA is one kind of phorbol ester known to activate PKC and PKD. Indicated from the top are the results of allowing the various samples to react with: (1) anti-PKD1/2 monoclonal antibody; (2) anti-fully active PKD1 polyclonal antibody; (3) anti-inactive PKD1 polyclonal antibody; (4) anti-sarcomeric-α-actinin monoclonal antibody; and (5) anti-H3 histone mouse monoclonal antibody. It is clear that the fully active PKD1 (phosphorylated PKD1) is localized in the membrane fraction of the TPA-treated NRC (Experiment 1).

FIG. 3. is a figure indicating the results of staining by immunofluorescence (confocal laser scanning) to investigate the intracellular distribution of α-actinin, fully active PKD1 (phosphorylated PKD1), and inactive PKD1 (non-phosphorylated PKD1) in TPA-treated NRC (FIG. A) and untreated NRC (FIG. 3B) (Experiment 2). Compared to FIG. 3B (untreated NRC), FIG. 3A (TPA-treated NRC) reveals the formation of sarcomere structures (cardiac hypertrophy state) from α-actinin localities, and it is evident that cardiac hypertrophy is induced by TPA treatment. It is also evident that TPA treatment only causes the fully active PKD1 (phosphorylated PKD1) to move to the sarcomere Z-disc, and to become localized there. The image in the right panels (Merged) is made by overlapping the image in the left panels and the image in the middle panels, and the more that yellow (red×green) present, the more the images coincide (the same applies to FIGS. 3 to 6, and 11 below).

FIG. 4 is a figure indicating the results of staining by immunofluorescence to investigate the intracellular distribution of α-actinin, fully active PKD1 (phosphorylated PKD1), and inactive PKD1 (non-phosphorylated PKD1) in NRC treated with norepinephrin (called NE below) that induces cardiac hypertrophy by acting on α-adrenergic receptors (Experiment 3). The NE includes proplanol (β-blocker) to suppress the action of β-adrenaline receptors in NRC. The development of sarcomere structures (cardiac hypertrophy state) by NE treatment was revealed. It is also evident that NE treatment only causes the fully active PKD1 (phosphorylated PKD1) to move to the sarcomere Z-disc, and to become localized there.

FIG. 5 is a figure indicating the results of staining by immunofluorescence to investigate the intracellular distribution of α-actinin, fully active PKD1 (phosphorylated PKD1), and inactive PKD1 (non-phosphorylated PKD1) in NRC treated with angiotensin II (called AngII below) (100 nM) which induces cardiac hypertrophy (Experiment 3). The formation of sarcomere structures (cardiac hypertrophy state) by AngII treatment was revealed. It is also evident that AngII treatment causes only the fully active PKD1 (phosphorylated PKD1) to move to the sarcomere Z-disc, and to become localized there.

FIG. 6 is a figure indicating the results of staining by immunofluorescence to investigate the intracellular distribution of α-actinin, fully active PKD1 (phosphorylated PKD1), and inactive PKD1 (non-phosphorylated PKD1) in NRC treated with LIF (leukemia inhibitory factor), which induces cardiac hypertrophy (Experiment 3). The formation of sarcomere structures (cardiac hypertrophy state) by LIF treatment was revealed. However, no movement of fully active PKD1 (phosphorylated PKD1) to the sarcomere Z-disc was observed.

FIG. 7A indicates the results of staining by immunofluorescence to investigate the intracellular distribution of α-actinin and fully active PKD1 (phosphorylated PKD1) after treating NRC with GF109203X, which is a selective PKC inhibitor, and then treating with the cardiac hypertrophy inducer: norepinephrine (NE) (+propranolol) (Experiment 3). It is evident that no cardiac hypertrophy state and no localization of fully active PKD1 (phosphorylated PKD1) on the sarcomere Z-disc were observed, and that GF109203X treatment inhibited NE (+propranolol)-induced cardiac hypertrophy state and localization of fully active PKD1 (phosphorylated PKD1) onto the sarcomere Z-disc (compare to FIG. 3). From these facts it is clear that the NE (+propranolol)-induced cardiac hypertrophy state, activation of PKD1, and translocation to the sarcomere Z-disc are dependent on PKC activation.

FIG. 7B indicates the results of staining by immunofluorescence to investigate the intracellular distribution of α-actinin and fully active PKD1 (phosphorylated PKD1) after treating NRC with GF109203X in the same way and then treating with the cardiac hypertrophy inducer: LIF (Experiment 3). In the same way as with independent treatment with LIF, a cardiac hypertrophy state was revealed, but translocation of fully active PKD1 (phosphorylated PKD1) to the sarcomere Z-disc was not observed. From these facts it is evident that the LIF induced cardiac hypertrophy state is not dependent on PKC activation, and that PKD1 activation and movement to the sarcomere Z-disc have no relationship to the LIF-induced cardiac hypertrophy.

FIG. 8 is a figure indicating the results of investigating the phosphorylation activity of PKD1 present in NRC or NRC treated with various types of drugs (Experiment 4). The abscissa indicates the type of NRC treated (or not treated) with the various types of drugs. Indicated from the left are: (1) untreated cells, (2) TPA-treated cells, (3) Norepinephrine (+propranolol) (NE) treated cells, (4) LIF-treated cells, (5) GF109203X-treated cells, (6) GF109203X+TPA-treated cells, (7) GF109203X+NE (+propranolol) treated cells, and (8) GF109203X+LIF-treated cells. The ordinate indicates the phosphorylation activity (%) of PKD1 present in the various cells. The phosphorylation activity (%) is the relative % when taking the phosphorylation activity of PKD1 present in untreated cells as 100%.

FIG. 9 is a figure indicating the results of investigating the changes in phosphorylation activity of intrinsic PKD1 when conducting NE (+propranolol) treatment of normal NRC, and NRC with inhibited activity caused by transient expression of various types of kinase dead PKCs (PKCα, PKCβI, PKCδ, PKCε, PKCξ) (Example 5). The abscissa indicates the types of NRC. Indicated from the left are: (1) natural NRC untreated by drugs, (2) NE-treated natural NRC, (3) NE-treated NRC-expressed kinase dead PKCα, (4) NE-treated NRC-expressed kinase dead PKCβI, (5) NE-treated NRC-expressed kinase dead PKCδ, (6) NE-treated NRC-expressed kinase dead PKCε, and (7) NE-treated NRC-expressed kinase dead PKCξ. The ordinate indicates the phosphorylation activity (%) of PKD1 present in the various cells. The phosphorylation activity (%) is the relative % when taking the phosphorylation activity of PKD1 present in untreated cells as 100%.

FIG. 10 is a figure indicating the results of an immunoprecipitation assay using the immunoprecipitation method and the Western blotting method to investigate the form of PKDε and PKD1 present in NE (+propranolol) treated NRC (Experiment 6).

FIG. 11A is a figure indicating the results of staining by immunofluorescence to investigate the intracellular distribution of α-actinin and GFP (specifically, GFP-PKD1 CA, or GFP) in NRC with transient expression by introducing GFP (green fluorescent protein) fused with constitutively active PKD1 (GFP-PKD1 CA) (lower panels), and GFP (upper panels) (Experiment 7 (1)). It is evident that only the NRC with transient expression of GFP-PKD1 CA (specifically, NRC having fully active PKD1 (phosphorylated PKD1)) formed sarcomere structures (hypertrophic state), and translocated to the sarcomere Z discs.

FIG. 11B is a figure indicating the results of staining by immunofluorescence to investigate the intracellular distribution of α-actinin and constitutively active PKCε (specifically, PKCε-CA) in NRC with transient expression by introducing PKCε-CA. Immunofluorescence was also observed for α-actinin and PKCε.

FIG. 12 is a figure indicating the results of staining by immunofluorescence to investigate the intracellular distribution of α-actinin in NRC with transient expression by introducing constitutively active PKCε (CA-PCKε) (panel A), in NRC with transient expression by introducing constitutively active PKCβI (CA-PKCβI) (panel B), and in NRC with transient expression by introducing constitutively active PKCδ (CA-PCKδ) (panel C) (Experiment 8). Moreover, panels D and E are figures indicating the results of staining by immunofluorescence to investigate the intracellular distribution of α-actinin in NRC with transient expression by introducing dominant negative PKCε (DN-PKCε) treated with NE (+propranolol) and LIF respectively (Experiment 8). The formation of sarcomere structures (cardiac hypertrophy state) is exhibited in panel A (NRC-expressed CA-PKCε), B (NRC-expressed CA-PKCβI), and E (NRC-expressed DN-PKCε+LIF-treated).

FIG. 13 is a figure indicating the results of investigating the level of expression of atrial natriuretic factor (called ANF below), which is a marker for cardiac hypertrophy, in various types of NRC. Indicated on the abscissa from the left are: (1) GFP introduced NRC+non-cardiac hypertrophy-induced (−) (negative control), (2) natural NRC+cardiac hypertrophy induced (NE treated), (3) NRC-expressed constitutively active PKCε (CA-PKCε)+non-cardiac hypertrophy-induced (−), (4) NRC-expressed constitutively active PKCβI (CA-PKCβI)+non-cardiac hypertrophy-induced (−), (5) NRC-expressed constitutively active PKCδ (CA-PKCδ)+non-cardiac hypertrophy-induced (−), (6) NRC-expressed kinase dead PKCε (KD-PKCε)+non-cardiac hypertrophy-induced (−), (7) NRC-expressed kinase dead PKCε (KD-PKCε)+cardiac hypertrophy-induced (NE treated), (8) NRC-expressed constitutively active PKD1 (CA-PKD1)+non-cardiac hypertrophy-induced (−), (9) NRC-expressed dominant negative PKD1 (DN-PKD1)+non-cardiac hypertrophy-induced (−), (10) NRC-expressed dominant negative PKD1 (DN-PKD1)+cardiac hypertrophy-induced (LIF treated), and (11) NRC-expressed dominant negative PKD1 (DN-PKD1)+cardiac hypertrophy-induced (NE treated). The ordinate indicates the level of ANF expression of the various cells. Further, the ANF expression of the various cells is indicated by the relative percentage when taking the level of ANF expression of the negative control as 1.

FIG. 14 indicates the domain structure of mouse ENH1 (mENH1) (enigma homologue 1), and the domain structures of mouse ENH2 (mENH2) (enigma homologue 2) and mouse ENH3 (mENH3) (enigma homologue 3), which are splice mutants having the LIM domain of the mENH1 deleted. All of the ENH molecules have PDZ domain at the N-terminal, and the mEHN1 has three LIM domains at the C-terminal. Both the “I” (internal stretch) and “T” (terminal stretch) are sequences with unknown functions.

FIG. 15 is a figure indicating the results of Example 10. The figure shows the NRC observed by the fluorescent antibody method using anti-FLAG antibody or anti-ANF antibody, the NRC is caused transient expression of ENH1 or ENH2, to which a FLAG epitope tag was added to the N terminal in advance, and treated with 20 nM TPA which is capable of inducing cardiac hypertrophy. The results of suppressing expression of ANF, which is a cardiac hypertrophy marker, demonstrated that the action of TPA to induce cardiac hypertrophy was suppressed only in the NRC into which ENH2 was introduced.

FIG. 16 is a conceptual diagram indicating a cardiac hypertrophy signal control model mediated through seven transmembrane-spanning heterotrimeric G protein-coupled receptor (called GPCR below) in cardiomyocytes. ENH1, PKCε, PKD1, and ENH2 have a correlative relationship with the cardiac hypertrophy signal control.

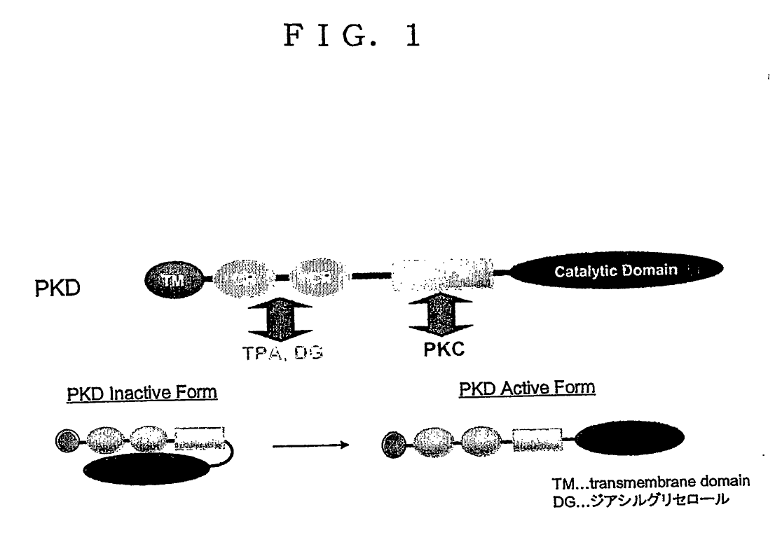

### DISCLOSURE OF THE INVENTION

The present inventors studied the role of protein kinase D1 (PKD1), as well as the interaction between PKD1 and protein kinase C_{ε} (PKC_{ε}) in cardiac hypertrophy signal transduction in cardiomyocytes. The details will be indicated in the experiments to be described later, but the main results were as follows: (1) PKD1 is notably expressed in cardiomyocytes, is fully activated (phosphorylated) by stimulus mediated through seven transmembrane-spanning heterotrimeric G protein-coupled receptor (GPCR) such as angiotensin II (AngII) and norepinephrine (NE), and moves into and is localized in sarcomere Z-discs; (2) the full activation (phosphorylation) of PKD1 is dependent on PKC_{ε} activation, and PKD1 interacts with PKC_{ε} within cardiomyocytes and is directly activated by PKC_{ε}; (3) when forcing the expression of fully activated PKD1 (phosphorylated PKD1) and active type PKC_{ε} respectively within cardiomyocytes, the cardiomyocytes indicate the same cardiac hypertrophy conditions as when treated by cardiac hypertrophy inducing agents, but after forcing the expression of inactive type PKD1 (non-phosphorylated PKD1), induced cardiac hypertrophy in cardiomyocytes was not observed even if conducting cardiac hypertrophy treatment. The results deeply implicate PKD1 as a signal factor in the signaling pathway generating cardiac hypertrophy (cardiac hypertrophy signaling pathway), and indicate that PKD1 induces cardiac hypertrophy by being directly activated by PKC_{ε}, and is a downstream factor of PKC_{ε}.

These results suggest as follows: it is possible to block the cardiac hypertrophy signaling pathway and suppress or reduce cardiac hypertrophy by inhibiting PKD1 activity in cardiomyocytes; a PKD1 inhibitor may be useful as cardiac hypertrophy suppressants and as medicinal agents to prevent or remedy heart disease; and conversely, it is possible to promote the onset of cardiac hypertrophy by increasing PKD1 activity in cardiomyocytes, and create and provide animal disease models of cardiac hypertrophy.

Moreover, it is well known that ENH1 is a scaffold protein that recruits and links the signal factors (PKC) that participate in cardiac hypertrophy signaling to the cardiomyocyte sarcomere Z-discs (Nakagawa N, et al., Biochem. Biophys. Res. Commun., 272(2), p. 505-512, 2000), but the present inventors discovered that spliced mutant ENH2 that lacks the LIM domain of ENH1 is an endogenous antagonist that suppresses cardiac hypertrophy signaling through the aforementioned ENH1. This result suggests that clinical applications as cardiac hypertrophy suppressants and as agents to prevent or remedy heart disease are possible, by suppressing and controlling the cardiac hypertrophy signaling in which ENH1 participate by forcing the expression of ENH2 in cardiomyocytes.

The present invention was completed based on the related knowledge, and first of all provides a pharmaceutical composition effective in suppressing cardiac hypertrophy related to the onset and development of heart diseases such as chronic cardiac failure. In more detail, the present invention provides a cardiac hypertrophy suppressant (pharmaceutical composition to suppress cardiac hypertrophy) that has as an active ingredient a substance that suppresses the functional expression in cardiomyocytes of PKD1 which is related to the cardiac hypertrophy signaling. Secondly, the present invention provides a pharmaceutical composition that can suppress the onset and development of various types of heart disease caused by cardiac hypertrophy by using a substance that suppresses the related functional expression of PKD1 in order to block or suppress the cardiac hypertrophy signaling. The present invention further provides a method to suppress cardiac hypertrophy and to prevent onset of cardiac hypertrophy, as well as a method to prevent or remedy the onset and development of various kinds of heart disease such as chronic cardiac failure that are caused by the aforementioned cardiac hypertrophy.

In addition, the present invention provides a method, based on the newly discovered mechanisms of generating cardiac hypertrophy, that screens and selects hypertrophy suppressants and the components effective to remedy or prevent cardiac diseases the onset and development of which are caused by cardiac hypertrophy; and provides pharmaceutical compositions having the related components as the active ingredients (pharmaceutical compositions to suppress cardiac hypertrophy, pharmaceutical compositions to prevent or remedy cardiac diseases caused by cardiac hypertrophy).

The present invention provides transgenic non-human animals, specifically, non-human animals that are disease models of cardiac hypertrophy in which cardiac hypertrophy is induced and promoted by transient expression of PKD1 in cardiomyocytes.

Concretely, the present invention contains the following forms.

I. Pharmaceutical Composition for Suppressing Cardiac Hypertrophy

(1) A pharmaceutical composition for suppressing cardiac hypertrophy, which comprises a substance that suppresses functional expression of PKD1 in cardiomyocytes an active ingredient.

(2) The pharmaceutical composition for suppressing cardiac hypertrophy according to (1), wherein the active ingredient is a substance that has an action to suppress PKD1 activity in cardiomyocytes.

(3) The pharmaceutical composition for suppressing cardiac hypertrophy according to (1) or (2), wherein the active ingredient is a substance that has an action to suppress phosphorylation of at least one of Ser-744, Ser-748, or Ser-916 of PKD1 derived from humans.

(4) The pharmaceutical composition for suppressing cardiac hypertrophy according to (1) or (2), wherein the active ingredient is nucleic acid having a base sequence that codes dominant negative PKD1 which has been controlled to be able to express in cardiomyocytes.

(5) The pharmaceutical composition for suppressing cardiac hypertrophy according to (4), wherein the nucleic acid is included in an expression vector.

(6) The pharmaceutical composition for suppressing cardiac hypertrophy according to (5), wherein the expression vector is a plasmid or a virus vector.

(7) The pharmaceutical composition for suppressing cardiac hypertrophy according to (5), wherein the expression vector is included within a virus particle or within a hollow nanoparticle.

(8) The pharmaceutical composition for suppressing cardiac hypertrophy according to (5), wherein the expression vector is comprised of liposomes.

(9) The pharmaceutical composition for suppressing cardiac hypertrophy according to (1) or (2), wherein the active ingredient is a substance that inhibits expression of PKD1 genes in cardiomyocytes.

(10) The pharmaceutical composition for suppressing cardiac hypertrophy according to (9), wherein the active ingredient is an antisense molecule, ribozyme or RNAi effector of PKD1.

(11) The pharmaceutical composition for suppressing cardiac hypertrophy according to (1) or (2), wherein the active ingredient is an antibody to PKD1 or to a fragment thereof that may be phosphorylated.

(12) A pharmaceutical composition for suppressing cardiac hypertrophy, which comprises nucleic acid having a base sequence that codes ENH2 an active ingredient.

(13) The pharmaceutical composition for suppressing cardiac hypertrophy according to (12), wherein the nucleic acid is included in an expression vector.

(14) The pharmaceutical composition for suppressing cardiac hypertrophy according to (13), wherein the expression vector is a plasmid or a virus vector.

(15) The pharmaceutical composition for suppressing cardiac hypertrophy according to (13), wherein the expression vector is included within a virus particle or within a hollow nanoparticle.

(16) The pharmaceutical composition for suppressing cardiac hypertrophy according to (13), wherein the expression vector is comprised of liposomes.

(17) The pharmaceutical composition for suppressing cardiac hypertrophy according to any of (1) through (16), wherein cardiac hypertrophy is caused by cardiac hypertrophy signal transduction through seven transmembrane-spanning heterotrimeric G protein-coupled receptors (GPCR) or epidermal growth factors (EGF) receptors.

(18) The pharmaceutical composition for suppressing cardiac hypertrophy according to any of (1) through (16), which further comprises gp130 receptor inhibitor.

II. Method to Suppress Cardiac Hypertrophy or to Prevent the Onset of Cardiac Hypertrophy

(1) A method to suppress cardiac hypertrophy or prevent onset of cardiac hypertrophy in a patient with cardiac hypertrophy or the preconditions thereof, which comprises administering the effective amount of a substance that suppresses functional expression of PKD1 in cardiomyocytes to the patient.

(2) The method according to (1), wherein the substance is a substance that has an action to suppress PKD1 activity in cardiomyocytes.

(3) The method according to (1) or (2), wherein the substance is a substance that has an action to suppress phosphorylation of at least one of Ser-744, Ser-748, or Ser-916 of PKD1 derived from humans.

(4) The method according to (1) or (2), wherein the substance is nucleic acid having a base sequence that codes dominant negative PKD1 which has been controlled to be able to express in cardiomyocytes.

(5) The method according to (4), wherein the nucleic acid is included in an expression vector.

(6) The method according to (5), wherein the expression vector is a plasmid or a virus vector.

(7) The method according to (5), wherein the expression vector is included within a virus particle or within a hollow nanoparticle.

(8) The method according to (5), wherein the expression vector is comprised of liposomes.

(9) The method according to (1) or (2), wherein the substance is a substance that inhibits expression of PKD1 genes in cardiomyocytes.

(10) The method according to (9), wherein the substance is an antisense molecule, ribozyme or RNAi effector of PKD1.

(11) The method according to (1) or (2), wherein the substance is an antibody to PKD1 or to a fragment thereof that may be phosphorylated.

(12) A method to suppress cardiac hypertrophy or to prevent onset of cardiac hypertrophy in a patient with cardiac hypertrophy or preconditions thereof, which comprises administering the effective amount of nucleic acid having a base sequence that codes ENH2 as an active ingredient.

(13) The method according to (12), wherein the nucleic acid is included in an expression vector.

(14) The method according to (13), wherein the expression vector is a plasmid or a virus vector.

(15) The method according to (13), wherein the expression vector is included within a virus particle or within a hollow nanoparticle.

(16) The method according to (13), wherein the expression vector is comprised of liposomes.

(17) The method according to any of (1) through (16), wherein cardiac hypertrophy is caused by cardiac hypertrophy signal transduction through GPCR or EGF receptor.

(18) The method according to any of (1) through (17), which further comprises gp130 receptor inhibitor.

III. Pharmaceutical Composition to Prevent or Remedy Heart Disease Caused by Cardiac Hypertrophy

(1) A pharmaceutical composition to prevent or remedy onset of heart disease caused by cardiac hypertrophy, which comprises a substance that suppresses functional expression of PKD1 in cardiomyocytes as an active ingredient.

(2) The pharmaceutical composition according to (1), wherein the active ingredient is a substance that has an action to suppress PKD1 activity in cardiomyocytes.

(3) The pharmaceutical composition according to (1) or (2), wherein the active ingredient is a substance that has an action to suppress phosphorylation of at least one of Ser-744, Ser-748, or Ser-916 of PKD1 derived from humans.

(4) The pharmaceutical composition according to (1) or (2) wherein the active ingredient is nucleic acid having a base sequence that codes dominant negative PKD1 which has been controlled to be able to express in cardiomyocytes.

(5) The pharmaceutical composition according to (4), wherein the nucleic acid is included in an expression vector.

(6) The pharmaceutical composition according to (5), wherein the expression vector is a plasmid or a virus vector.

(7) The pharmaceutical composition according to (5), wherein the expression vector is included within a virus particle or within a hollow nanoparticle.

(8) The pharmaceutical composition according to (5), wherein the expression vector is comprised of liposomes.

(9) The pharmaceutical composition according to (1) or (2), wherein the active ingredient is a substance that inhibits expression of PKD1 genes in cardiomyocytes.

(10) The pharmaceutical composition according to (9), wherein the active ingredient is an antisense molecule, ribozyme or RNAi effector of PKD1.

(11) The pharmaceutical composition according to (1) or (2), wherein the active ingredient is an antibody to PKD1 or to a fragment thereof that may be phosphorylated.

(12) A pharmaceutical composition to prevent or remedy the onset of heart disease caused by cardiac hypertrophy, which comprises nucleic acid having a base sequence that codes ENH2 as an active ingredient.

(13) The pharmaceutical composition according to (12), wherein the nucleic acid is included in an expression vector.

(14) The pharmaceutical composition according to (13), wherein the expression vector is a plasmid or a virus vector.

(15) The pharmaceutical composition according to (13), wherein the expression vector is included within a virus particle or within a hollow nanoparticle.

(16) The pharmaceutical composition according to (13), wherein the aforementioned expression vector is comprised of liposomes.

(17) The pharmaceutical composition according to any of (1) through (16), wherein cardiac hypertrophy is caused by cardiac hypertrophy signal transduction through GPCR or EGF receptor.

(18) The pharmaceutical composition according to any of (1) through (17), wherein the diseases caused by cardiac hypertrophy are heart failure, ischemic heart disease or arrhythmia.

(19) The pharmaceutical composition according to (1) through (16) and (18), which further comprises gp130 receptor inhibitor.

IV. Method to Prevent or Remedy Heart Disease Caused by Cardiac Hypertrophy

(1) A method to prevent or remedy onset of diseases caused by cardiac hypertrophy in a patient with cardiac hypertrophy or the preconditions thereof, which comprises administering to the patient the effective amount of a substance that suppresses functional expression of PKD1 in cardiomyocytes.

(2) The method according to (1), wherein the substance is a substance that has an action to suppress PKD1 activity in cardiomyocytes.

(3) The method according to (1) or (2), wherein the substance is a substance that has an action to suppress phosphorylation of at least one of Ser-744, Ser-748, or Ser-916 of PKD1 derived from humans.

(4) The method according to (1) or (2), wherein the substance is nucleic acid having a base sequence that codes dominant negative PKD1 which has been controlled to be able to express in cardiomyocytes.

(5) The method according to (4), wherein the nucleic acid is included in an expression vector.

(6) The method according to (5), wherein the expression vector is a plasmid or a virus vector.

(7) The method according to (5), wherein the expression vector is included within a virus particle or within a hollow nanoparticle.

(8) The method according to (5), wherein the expression vector is comprised of liposomes.

(9) The method according to (1) or (2), wherein the substance is a substance that inhibits expression of PKD1 genes in cardiomyocytes.

(10) The method according to (9), wherein the substance is an antisense molecule, ribozyme or RNAi effector of PKD1.

(11) The method according to (1) or (2), wherein the substance is an antibody to PKD1 or to a fragment thereof that may be phosphorylated.

(12) A method to prevent or remedy the onset of diseases caused by cardiac hypertrophy in a patient with cardiac hypertrophy or preconditions thereof, which comprises administering the effective amount of nucleic acid having a base sequence that codes ENH2 to the patient.

(13) The method according to (14), wherein the nucleic acid is included in an expression vector.

(14) The method according to (13), wherein the expression vector is a plasmid or a virus vector.

(15) The method according to (13), wherein the expression vector is included within a virus particle or within a hollow nanoparticle.

(16) The method according to (13), wherein the expression vector is comprised of liposomes.

(17) The method according to any of (1) through (16), wherein cardiac hypertrophy is caused by cardiac hypertrophy signal transduction through GPCR or EGF receptor.

(18) The Method according to any of (1) through (17), wherein the cardiac diseases caused by cardiac hypertrophy are heart failure, ischemic heart disease or arrhythmia.

(19) The method according to any of (1) through (16) and (18), which comprises administration of gp130 receptor inhibitor, as well as the substance that suppresses functional expression of PKD1 in cardiomyocytes.

V. Method to Block or Suppress Cardiac Hypertrophy Signal Transduction

(1) A method to block hypertrophy signal transduction, which comprises administering the effective amount of a substance that inhibits functional expression of PKD1 to cardiomyocytes.

(2) The method according to (1), wherein the substance is a substance that has an action to suppress PKD1 activity in cardiomyocytes.

(3) The method according to (1) or (2), wherein the substance is a substance that has an action to suppress phosphorylation of at least one of Ser-744, Ser-748, or Ser-916 of PKD1 derived from humans.

(4) The method according to (1) or (2), wherein the substance is nucleic acid having a base sequence that codes dominant negative PKD1 which has been controlled to be able to express in cardiomyocytes.

(5) The method according to (4), wherein the nucleic acid is included in an expression vector.

(6) The method according to (5), wherein the expression vector is a plasmid or a virus vector.

(7) The method according to (5), wherein the expression vector is included within a virus particle or within a hollow nanoparticle.

(8) The method according to (5), wherein the expression vector is comprised of liposomes.

(9) The method according to (1) or (2), wherein the substance is a substance that inhibits expression of PKD1 genes in cardiomyocytes.

(10) The method according to (9), wherein the substance is an antisense molecule, ribozyme or RNAi effector of PKD1.

(11) The method according to (1) or (2), wherein the substance is an antibody to PKD1 or to a fragment thereof that may be phosphorylated.

(12) A method to block cardiac hypertrophy signal transduction, which comprises administering an effective amount of nucleic acid having a base sequence that codes ENH2 to cardiomyocytes.

(13) The method according to (12), wherein the nucleic acid is included in an expression vector.

(14) The method according to (13), wherein the expression vector is a plasmid or a virus vector.

(15) The method according to (13), wherein the expression vector is included within a virus particle or within a hollow nanoparticle.

(16) The method according to (13), wherein the expression vector is comprised of liposomes.

(17) The method according to any of (1) through (16), wherein cardiac hypertrophy is caused by hypertrophy signal transduction through GPCR or EGF receptor.

(18) The method according to any of (1) through (16), which comprises further administering a gp130 receptor inhibitor.

VI. Transgenic Non-Human Animal

(1) A transgenic non-human animal which a constitutively active PKD1 is transiently expressed in cardiomyocytes.

(2) The transgenic non-human animal according to (1), wherein the constitutively active PKD1 is at least one selected from human derived PKD1 with the PH domain deleted, human derived PKD1 with the serines of position 744 and position 748 of the amino acid sequence substituted with glutamic acids, and mouse derived PKD1 with the serines of position 744 and position 748 of the amino acid sequence substituted with glutamic acids.

(3) The transgenic non-human animal according to (1) or (2), which is an animal model of cardiac hypertrophy.

(4) The transgenic non-human animal, wherein the dominant negative PKD1 is transiently expressed in cardiomyocytes.

(5) The transgenic non-human animal according to (4), wherein the dominant negative protein kinase D1 is at least one selected from human derived PKD1 with the lysine of position 612 of the amino acid sequence substituted with tryptophan, human derived PKD1 with the lysine of position 618 of the amino acid sequence substituted with asparagine, human derived PKD1 with the aspartic acid of position 733 of the amino acid sequence substituted with alanine, human derived PKD1 with the serines of position 738 and position 742 of the amino acid sequence substituted with alanines, mouse derived PKD1 with the lysine of position 618 of the amino acid sequence substituted with methionine, and mouse derived PKD1 with the serines of position 744 and position 748 of the amino acid sequence substituted with alanines.

VII. Screening Method

(1) A method for screening a cardiac hypertrophy suppressant comprising the following steps:

(a) bringing a test substance into contact with cells that can express PKD1;

(b) measuring the levels of expression of PKD1 in the aforementioned cells, and comparing with the level of PKD1 expression in contrast cells that were not brought into contact with the test substance; and

(c) based on the comparative results of (b) above, selecting as a cardiac hypertrophy suppressant the test substance which, when brought into contact with cells, lowered the level of expression of PKD1 as compared to the contrast cells.

(2) A method for screening a cardiac hypertrophy suppressant comprising the following steps:

(a) bringing a PKD1 activator and a test substance into contact with cells that can express PKD1;

(b) measuring the activity of PKD1 in the aforementioned cells, and comparing with the activity corresponding to the above in contrast to cells that were not brought into contact with the test substance; and

(c) based on the comparative results of (b) above, selecting as a cardiac hypertrophy suppressant the test substance which, when administered to cells, lowered the activity of PKD1 as compared to the contrast cells.

(3) A method for screening a cardiac hypertrophy suppressant comprising the following steps:

(a) bringing a test substance and a cardiac hypertrophy inducer that stimulates GPCR or EGF receptor into contact with cardiomyocytes;

(b) measuring the PKD1 activity, localization of phosphorylated PKD1 in sarcomere Z-discs, or the intermolecular distance of PKC_{ε} and PKD1 in the aforementioned cardiomyocytes, and comparing with the corresponding activity, localization or intermolecular distance in contrast to cardiomyocytes that were brought into contact with hypercardia inducer only; and

(c) based on the comparative results of (b) above, selecting as cardiac hypertrophy suppressants the test substance administered to the cardiomyocytes that lowered the activity of PKD1, or lowered the localization of phosphorylated PKD1 in sarcomere Z-discs, or increased the intermolecular distance of PKC_{ε} and PKD1 compared to the contrast cardiomyocytes.

(4) A method for screening a cardiac hypertrophy suppressant comprising the following steps:

(a) bringing a test substance into contact with cardiomyocytes that can express constitutively active PKC_{ε} or constitutively active PKD1;

(b) measuring the PKD1 activity, localization of phosphorylated PKD1 in sarcomere Z-discs, or the intermolecular distance of PKC_{ε} and PKD1 in the aforementioned cardiomyocytes, and comparing with the activity, localization or intermolecular distance corresponding to the above in contrast cardiomyocytes that were not brought into contact with the test substance; and

(c) based on the comparative results of (b) above, selecting as a cardiac hypertrophy suppressant the test substance administered to the cardiomyocytes that lowered the activity of PKD1, or lowered the localization of phosphorylated PKD1 in sarcomere Z-discs, or increased the intermolecular distance of PKC_{ε} and PKD1 compared to the contrast cardiomyocytes.

(5) The method for screening according to (4), wherein the constitutively active PKC_{ε} has region 156 to 162 of the amino acid sequence deleted in human derived PKC_{ε}.

(6) The method for screening according to (4), wherein the constitutively active PKD1 is at least one selected from human derived PKD1 with the serines at position 744 and position 748 of the amino acid sequence is substituted with glutamic acids, human derived PKD1 with the PH domain deleted, and mouse derived PKD1 with the serines of position 744 and position 748 of the amino acid sequence substituted with glutamic acids.

(7) The method for screening to any of (3) to (6), which is a method for selecting a substance having an action to suppress phosphorylation of at least one of Ser-744, Ser-748, or Ser-916 of human derived PKD1 from the test substances.

(8) A method for screening cardiac hypertrophy suppressants comprising the following steps:

(a) administering a test substance to transgenic non-human animals according to any of IV (1) to (3);

(b) measuring the degree of cardiac hypertrophy of the aforementioned non-human animals, and comparing with the extent of cardiac hypertrophy of contrast transgenic non-human animals that were not administered the test substances; and

(c) based on the comparative results of (b) selecting as cardiac hypertrophy suppressants the test substances that reduce or suppress cardiac hypertrophy.

(9) The method for screening according to any of (1) through (8), which is a method for acquiring active ingredients for pharmaceutical compositions to prevent or remedy heart disease caused by cardiac hypertrophy.

(10) The method for screening according to (9), wherein the heart disease caused by cardiac hypertrophy is heart failure, ischemic heart disease or arrhythmia.

(VIII) Use

(1) Use of a substance that suppresses functional expression of PKD1 in cardiomyocytes, or of nucleic acid having a base sequence to code ENH2, for manufacturing pharmaceutical compositions to suppress cardiac hypertrophy.

(2) Use according to (1), wherein the substance that suppresses functional expression of PKD1 in cardiomyocytes is a substance that suppresses the activity of PKD1 in cardiomyocytes or a substance that prevents expression of PKD1 genes in cardiomyocytes.

(3) Use of a substance that suppresses functional expression of PKD1 in cardiomyocytes, or of nucleic acid having a base sequence to code ENH2, for manufacturing pharmaceutical compositions to prevent or remedy onset of heart diseases caused by cardiac hypertrophy.

(4) Use according to (3), wherein the substance that suppresses functional expression of PKD1 in cardiomyocytes is a substance that suppresses the activity of PKD1 in cardiomyocytes or a substance that prevents expression of PKD1 genes in cardiomyocytes.

### BEST MODE FOR CARRYING OUT THE INVENTION

1. Pharmaceutical Composition to Suppress Cardiac Hypertrophy

Cardiac hypertrophy is caused by increased load based on exercise, and disease factors such as increased pressure load based on hypertension, increased volume load based on valvular disorders, and increased load based on diseases of unknown cause. The cardiac hypertrophy of the present invention means the latter, specifically, myocardial disease conditions, such as compensatory hypertrophy of the heart and hypertrophic myocardial disease, in which the volume of the heart has increased beyond the range of physiological hypertrophy, based on various stresses such as hemodynamic overload and liquid factors by the disease condition.

Differences in hypertrophy of various parts of the heart, such as left ventricular hypertrophy, right ventricular hypertrophy, bilateral ventricular hypertrophy, and atrial hypertrophy may arise depending on the part where cardiac load is applied, but these types of hypertrophy are not particularly distinguished in the present invention. Moreover, if the overload applied to the heart is pressure load, there is a tendency for the wall thickness to increase notably and for the inner chamber to become deformed or narrowed (concentric hypertrophy); and if the overload applied to the heart is volume load, there is a tendency for the inner chamber to expand without that much increase in wall thickness (eccentric hypertrophy). In the present invention, however, these types are not particularly distinguished. Positively speaking, the present invention may be suitably used on the former, concentric hypertrophy, which indicates a shape with increased wall thickness.

Moreover, the cardiac hypertrophy targeted by the present invention is induced via signal transduction generated through activation of G-proteins mediated by 7-pass membrane G protein-coupled receptors (GPCR), or through activation of receptor tyrosine kinase mediated by epidermal growth factor receptors (EGF receptors). Further, it appears that protein synthesis is promoted in cardiomyocytes and hypertrophy is induced when seven transmembrane-spanning heterotrimeric G protein-coupled receptors (GPCR) are stimulated by liquid factors such as angiotensin II (AngII), endothelin-1, and norepinephrine (NE), thereby activating immediately downstream heterotrimeric G-proteins, activating Ca²⁺/inositol turnover, and activating the cardiac hypertrophy-related transcription factors.

The pharmaceutical compositions for suppressing cardiac hypertrophy of the present invention comprise as active ingredients substances that suppress the functional expression of PKD1 in cardiomyocytes. Here, suppression includes both 100% suppression (inhibition) of the functional expression of PKD1, and reduction of the original function of PKD1 without 100% inhibition. The substances may be ones that result in suppression of the functional expression of PKD1 in cardiomyocytes, and the following may be cited as examples: substances that suppress the expression or production of PKD1 in cardiomyocytes, substances that block or suppress PKD1 activation signals in cardiomyocytes, and substances that suppress the activation (including phosphorylation) of PKD1 in cardiomyocytes.

For example, substances that suppress the transcription, RNA processing, transfer, translation and/or stability of PKD1 genes during the expression or production of PKD1 in cardiomyocytes may be cited as examples of substances that suppress the expression or production of PKD1 in cardiomyocytes. Concretely, antisense molecules, ribozymes and RNAi effectors that can hybridize with the base sequence of genes that code PKD1 and can suppress the transcription, RNA processing, transfer, translation and/or stability thereof may be cited as examples of such substances.

The antisense molecules used in the present invention are designed to bond to the promoter or other control region, exon, intron, or exon-intron boundary of PKD1 gene. Many effective antisense molecules are designed to hybridize with the intron/exon splice connection part. Consequently, in order to hybridize with a region within 50 to 200 bases of the intron/exon splice connection part of the PKD1 gene, the antisense molecule of the present invention preferably has a substantially complementary base sequence to the aforementioned region.

Ribozymes are RNA-protein complexes, and are substances that manifest functions to inhibit translation to proteins and to suppress functional expression of gene by site-specific bonding to the target gene (mRNA) and cutting. The ribozymes used in the present invention are designed to hybridize with any region of the mRNA transcribed from the PKD1 gene (DNA) (for example, to have substantially complementary base sequences to the region); to cut the phosphate ester residue of oligonucleotide within the hybridized target region; and to inhibit translation to PKD1.

RNAi effectors are substances that hybridize to the upstream region of PKD1 DNA or mRNA, and specifically suppress the expression of PKD1 genes by functioning RNAi (RNA interference). As RNAi effectors, siRNA (small interfering RNA), stRNA (small temporally regulated RNA), and shRNA (short hairpin RNA) may be cited. The RNAi technology utilizing RNAi effectors and methods thereof are described in detail in Kazushi Taiyoshi, et al, ed. “RNAi Experimental Protocol”, YODOSHA CO. LTD., 2003, and the contents of the document are hereby incorporated by reference.

Moreover, nucleic acids that code for dominant negative PKD1 controlled to be capable of expression in cardiomyocytes may be understood as substances that suppress expression or production of PKD1 in cardiomyocytes. For example, it is known that mutation in the ATP binding position of PKD1 (for example, Lys-residue of position 612 in human derived PKD1, and Lys-residue of position 618 in mouse derived PKD1) predominantly suppresses the kinase function thereof. Concretely, the following may be cited as examples of dominant negative PKD1: human derived PKD1 with the lysine of position 618 (Lys-618) of the amino acid sequence substituted with asparagine (K618N PKD1), human derived PKD1 with the lysine of position 612 (Lys-612) of the amino acid sequence substituted with tryptophan, human derived PKD1 with the aspartic acid of position 733 (Asp-733) of the amino acid sequence substituted with alanine, human derived PKD1 with the serines of positions 738 and 742 of the amino acid sequence substituted with alanines, mouse derived PKD1 with the lysine of position 618 (Lys-618) of the amino acid sequence substituted with methionine, and mouse derived PKD1 with the serines of positions 744 (Lys-744) and 748 (Lys-748) of the amino acid sequence substituted with alanines.

The nucleic acids coding for dominant negative PKD1 is normally used in a state operably bound to the functional DNA sequence required for expression of the related nucleic acids in cardiomyocytes. Here, functional DNA means the control region or regulatory element necessary in order for nucleic acids coding for dominant negative PKD1 to be expressed in cardiomyocytes, and polyadenylated signals, upstream sequence domains, promoters, enhancers or terminators may be cited as examples. Concrete examples of promoters include SV40 early promoter, mouse mammary tumor virus LTR promoter, adenovirus major late promoter (Ad MLP), simple herpes promoter, CMV promoter (for example, CMV initial promoter, Raus sarcoma virus (RSV) promoter), myosin light chain 2 promoter, α-actin promoter, troponin 1 promoter, Na⁺/Ca²⁺ substituted promoter, dystrophin promoter, creatine kinase promoter, α7 integrin promoter, brain natriuretic peptide promoter, αB-crystallin/small heat shock protein promoter, α-myosin heavy chain promoter, and ANF promoter. It is more preferable that these promoters are ones that can be expressed tissue specifically in cardiomyocytes.

Here, “operably bound” means that the nucleic acid that codes for dominant negative PKD1 is present in a state that can be expressed in cardiomyocytes irrespective of the binding position and direction with respect to the aforementioned various types of functional DNA sequences.

Based on a mechanism of suppressing cardiac hypertrophy different from the above, the present invention provides as a substance to suppress and control the cardiac hypertrophy signal cascade in cardiomyocytes a nucleic acid having a base sequence that codes for ENH2 controlled to be capable of expression in cardiomyocytes. In the same way as the above, the nucleic acid in question is normally used in a state operably bound to a functional DNA sequence necessary for the expression of the nucleic acid in cardiomyocytes. Base sequences codig for ENH2 have already been widely known by Nakagawa N. et al. (Nakagawa N, Hoshijima M, Oyasu M, Saito N, Tanizawa K, Kuroda S., ENH containing PDZ and LIM domains, heart/skeletal muscle-specific protein, associates with cytoskeletal proteins through the PDZ domain. Biochem. Biophys. Res. Commun., 2000 Jun. 7, 272 (2), p. 505-12.), the contents of the literature are hereby incorporated by reference.

These nucleic acids are preferably provided in forms suitable for administration to test subjects, preferably, to mammals including humans.

Expression vectors for genetic therapies may be cited as the forms in question. The related expression vectors for genetic therapies may be prepared and prescribed by well-known methods in this field corresponding to the desired administration route. Methods to prevent release or absorption of the target nucleic acids from the expression vector until the expression vector reaches the target organ (heart, cardiomyocytes) are well known in this field, and can be applied to the present invention in the same way. The expression vectors may also be used in a state forming a complex with other vehicles (for example, molecules with a lipid base such as liposome, aggregate proteins, or transporter molecules). These expression vectors may be transmitted to the cardiomyocytes by injection in the coronary artery or coronary sinus (called intra-coronary transmission through the coronary artery, intra-coronary artery transmission, or intra-artery transmission) (For example, refer to U.S. Pat. Nos. 5,792,453 and 6,100,242. The contents of these literature are quoted and incorporated in the present invention.).

Plasmid vectors and virus vectors are included in the expression vectors of the present invention. With virus vectors, the nucleic acid coding for dominant negative PKD1 or the nucleic acid coding for ENH2 is used in a state operably bound with the functional DNA sequence necessary for expression in cardiomyocytes, and in a state encloded in a viral particle. Any of the virus vectors well known in the field may be optionally used, and examples include adenovirus, retrovirus, adeno-associated virus, vaccinia virus, herpes virus, and polyoma virus.

Instead of the aforementioned virus vectors, it is also possible to use hollow nanoparticles, in which bio-recognition molecules that can specifically recognize heart tissue or cardiomyocytes are introduced into a substance having the ability to form particles. The hollow nanoparticles in question are well known in the field as transporters for introducing a desired substance into targeted cells or tissues. A detailed description of the hollow nanoparticles in question is given in, for example, Japanese Laid-open Patent No. 2001-316298, and the contents of the gazette are incorporated hereto by reference.

The following may be cited as examples of substances that suppress activation of PKD1 in cardiomyocytes: substances that suppress release of the CR domain from the catalyst domain of PKD1, substances that suppress phosphorylation of PKD1 (for example, substances that suppress phosphorylation of at least one of Ser-744, Ser-748 or Ser-916 of human derived PKD1), substances that suppress formation of a complex of PKCε and PKD1, substances that selectively bond to the substrate-binding sites of PKD1 and suppress enzyme activity, and substances that suppress sarcomere Z-discs localization of PKD1 (for example, substances that suppress recruiting of PKD1 to sarcomere Z-discs, or substances that suppress PKD1 bonding to scaffolds, such as ENH1, on sarcomere Z-discs).

Concretely, antibodies that react with PKD1 or any parts thereof may be cited as the substances that suppress activation of PKD1 in cardiomyocytes. The antibodies may be either polyclonal antibodies or monoclonal antibodies. Preferably, the antibodies are monoclonal antibodies. Methods to prepare polyclonal antibodies and monoclonal antibodies are well known in the field, and the antibodies of the present invention may be prepared accordingly (For example, refer to Harlow and Lane, Antibodies; A Laboratory manual, Cold Spring Harbor Laboratory, 1988; and U.S. Pat. No. 4,196,265. The contents of this literature is incorporated hereto by reference.). Concretely, the following may be cited as antibodies preferably used in the present invention: anti-PKD1 antibody (this means both polyclonal antibody and monoclonal antibody), antibody to the CR domain of PKD1 (anti-PKD1/CR domain antibody), antibody to PH domain of PKD1 (anti-PKD1/PH domain antibody), antibody to the phosphorylation region of PKD1 (for example, in the case of human derived PKD1, (1) antibody to peptide fragment of PKD1 comprising at least one of Ser-744, Ser-748, or Ser 916, or (2) antibody to a peptide fragment of PKD1 comprising at least one of phosphorylated Ser-744, Ser-748, or Ser 916), antibody to catalyst region of PKD1, anti-inactive PKD1 antibody (stabilizes inactive PKD1), and anti-fully active PKD1 antibody (bonds to PKD1 in the activated state, and suppresses enzyme activity by steric hindrance.)

The pharmaceutical composition for suppressing cardiac hypertrophy of the present invention may also comprises pharmaceutically acceptable carriers and additives corresponding to the form or administration route of preparation, in addition to an effective amount of the active ingredient, which is a substance that suppresses the functional expression of PKD1 in cardiomyocytes, or nucleic acid that codes ENH2. The pharmaceutical composition may be administered by the desired mode, for example by oral administration, intravenous administration, intramuscular administration, hypodermic administration, transpulmonary administration, transnasal administration, transintestinal administration, intraperitoneal administration or administration in coronary artery or coronary sinus. The pharmaceutical composition was prepared into solid administration forms such as tablets, pills, bulk drug, powder, granules and capsules, etc.; liquid administration forms such as solutions, suspensions, emulsions, syrups, liposome preparations, injectable agents, intravenous agents, drip agents, and elixirs, etc.; and external dosing forms such as patches, ointments, creams and sprays.

Examples of carriers used to formulate these pharmaceutical compositions (pharmaceutical preparations), as commonly used corresponding to the form of administering the preparation, include fillers, diluents, binders, moisturizers, disintegrators, disintegration suppressants, absorption promoters, lubricants, dissolution supplements, buffers, emulsifiers, and suspending agents. Examples of additives, as commonly used corresponding to the form of administering the preparation, include stabilizers, preservatives, buffers, extenders, chelates, pH adjusters, surfactants, colorants, fragrances, flavors, and sweeteners.

The pharmaceutical composition for suppressing cardiac hypertrophy of the present invention may also comprises substances that block the cardiac hypertrophy signaling pathway but are not mediated through stimulus for seven transmembrane-spanning heterotrimeric G protein-coupled receptors (GPCR) or epidermal growth factors (EGF) receptors, for example, substances that block the cardiac hypertrophy signaling pathway mediated through gp130 receptors (gp130 receptor inhibitor) such as cytokine receptor blockers and LIF inhibitors, in addition to an active ingredient which is substances that suppress the functional expression of PKD1 in cardiomyocytes or nucleic acids that codes ENH2.

The amount of active ingredient to be contained in the aforementioned pharmaceutical composition and the dosage thereof are not particularly limited, and may be suitably selected in a range corresponding to the desired therapeutic effect, administration manner, therapy period, age or sex of the patient, and other conditions. The dosage varies depending on the administration route, but normally dosage in the range of approximately 0.1 pg to 100 mg/kg may be administered by calculating the amount of active ingredient per dose.

II. Method to Suppress Cardiac Hypertrophy, Method to Prevent the Onset of Cardiac Hypertrophy and the Development thereof

The method to suppress cardiac hypertrophy of the present invention can be achieved by administering a patient with cardiac hypertrophy or preconditions thereof with the effective amount of a substance that suppresses the functional expression in cardiomyocytes of PKD1, or of nucleic acid that codes for ENH2. In addition, for a patient with cardiac hypertrophy or preconditions thereof, the method in question may be effectively used as a method to prevent onset of cardiac hypertrophy and development thereof.

Here, the same substances described in section I. may be cited as substances that suppresses the functional expression in cardiomyocytes of PKD1, or nucleic acids that code for ENH2. These substances may be used in the form of pharmaceutical compositions at a dose effective to suppress cardiac hypertrophy together with pharmaceutically acceptable carriers and other additives. The formulations, administration routes, and modes of administration, and dosage of the pharmaceutical composition are as previously described in section I.

Humans and other mammals may be cited as test subjects targeted for administration. The mammals in question are not particularly limited, and, concretely, may include rats, mice, hamsters, guinea pigs, dogs, monkeys, cows, horses, sheep, goats, and pigs, etc.

III. Pharmaceutical Composition to Prevent or Remedy Heart Diseases Caused by Cardiac Hypertrophy

As previously described, according to the Framingham Heart Study, etc., it is well known that cardiac hypertrophy is a risk factor inviting heart failure, ischemic heart diseases such as angina pectoris and myocardial infarction, and cardiovascular diseases such as arrhythmia. Consequently, based on the fact that the previously described substances that suppresses the functional expression in cardiomyocytes of PKD1 or nucleic acids that code for ENH2 suppress hypercardia in cardiomyocytes, the same substances can be used as drugs to effectively prevent or remedy onset of various types of heart disease caused by cardiac hypercardia.

As described above, heart failure (congestive heart failure, acute left-sided heart failure, acute pulmonary heart failure, contractive failure such as cardiogenic shock (attack of myocardial infarction, bradycardia, tachycardia), hypertrophic myocardosis, amyloidosis, systolic pericarditis, and expansive failure such as pericardial tamponade), ischemic heart disease (angina pectoris, myocardial infarction) and arrhythmia may be cited as examples of heart diseases caused by cardiac hypertrophy.

The same substances as those described in section I. may be cited as the substances that suppress the functional expression in cardiomyocytes of PKD1 or as the nucleic acids that code for ENH2, which are to be used as the active ingredients.

These substances may be used in the form of pharmaceutical compositions at a dose effective to prevent or remedy heart diseases caused by cardiac hypertrophy together with pharmaceutically acceptable carriers and other additives. The pharmaceutical composition of the present invention may contain well-known therapeutic drugs for heart disease as necessary. The therapeutic drugs for heart disease are not particularly limited, but β-blockers, anti-hypertensive agents, cardiotonic agents, anti-thrombosis agents, vasodilators, endothelia receptor blockers, calcium channel blockers, phosphodiesterase inhibitors, AngII receptor blockers, cytokine receptor blockers, and gp130 receptor inhibitors may be cited as examples.

The formulations and administration routes, modes of administration and dosage of the pharmaceutical compositions are as previously described in section I.

IV. Method to Prevent or Remedy Heart Disease Caused by Cardiac Hypertrophy

The method to prevent or remedy heart disease caused by cardiac hypertrophy of the present invention may be carried out by administering to test subjects with heart diseases caused by cardiac hypertrophy or the preconditions thereof the effective amount of a substance that suppresses the functional expression in cardiomyocytes of PKD1 or of nucleic acid that codes for ENH2. The method in question may be effectively used as a method to prevent cardiac hypertrophy from developing into heart disease for a test subject with cardiac hypertrophy.

Here, the same substances as those described in section I. may be cited as the substances that suppress the functional expression of PKD1 in cardiomyocytes or the nucleic acids that code for ENH2. The substances in question may be used in the form of pharmaceutical compositions at a dose effective to prevent or remedy heart diseases caused by cardiac hypertrophy together with pharmaceutically acceptable carriers and other additives. The formulations, administration routes, modes of administration, and dosage of the pharmaceutical composition are as previously described in section I. The pharmaceutical compositions of the present invention may be coadministered with well-known therapeutic drugs for heart disease as necessary. The therapeutic drugs for heart disease in question are not particularly limited, but β-blockers, anti-hypertensive agents, cardiotonic agents, anti-thrombosis agents, vasodilators, endothelia receptor blockers, calcium channel blockers, phosphodiesterase inhibitors, AngII receptor blockers, cytokine receptor blockers, and gp130 receptor inhibitors may be cited as examples.

The same heart failure, ischemic heart diseases such as angina pectoris and myocardial infarction, and cardiovascular diseases such as arrhythmia described in section III. may be cited as the types of heart disease caused by cardiac hypertrophy. The same human or other mammals (rats, mice, hamsters, guinea pigs, dogs, monkeys, cows, horses, sheep, goats, and pigs, etc.) described in section II. may be cited as the test subjects targeted for treatment.

V. Method to Block Cardiac Hypertrophy Signal Transduction

The previously described method to suppress cardiac hypertrophy and method to prevent or remedy heart disease caused by cardiac hypertrophy may be carried out by suppressing the functional expression of PKD1 in cardiomyocytes or by the transient expression of ENH2 within cardiomyocytes. This is based on the fact that suppressing the functional expression of PKD1 in cardiomyocytes or the transient expression of ENH2 inhibits either cardiac hypertrophy signal transduction mediated though GPCR or EGF receptor or cardiac hypertrophy signal transduction involving ENH1.

For this reason, from a separate point of view, the present invention provides a method to block or suppress the cardiac hypertrophy signal transduction in cardiomyocytes. The cardiac hypertrophy signal transduction targeted here is one mediated directly through GPCR or EGF receptor. The blocking or suppression of the cardiac hypertrophy signaling may be carried out by administering to the test substance the effective amount of a substance that inhibits functional expression of PKD1 in cardiomyocytes or of a nucleic acid that codes for ENH2. The test substance in question may be cardiomyocytes or tissues having the same, or may be cultured cardiomyocytes, cultured heart tissue, or cardiomyocytes or heart tissue present in a living body. Here, the source of the cardiomyocytes or heart tissue is not particularly at issue, and humans or other mammals (rats, mice, hamsters, guinea pigs, dogs, monkeys, cows, horses, sheep, goats, and pigs, etc.) may be broadly cited. The same substances and amounts thereof previously described in section I. may be cited as the substances that suppress functional expression of PKD1 or a nucleic acid that code for ENH2 and the amounts thereof that are to be administered to the test subjects.

Substances that block cardiac hypertrophy signal pathway mediated through gp130 receptors such as cytokine blockers, etc. (for example, gp130 receptor inhibitors) may be administered in combination with substances that suppress functional expression of PKD1 or nucleic acid that code for ENH2. By doing this, it is possible to jointly block cardiac hypertrophy signal pathways different from those mediated through GPCR or EGF receptor.

VI. Transgenic Non-Human Animals

The present invention provides transgenic non-human animals constructed to express protein related to PKD1 in the cells of non-human animals.

Here, mammals such as rats, mice, hamsters, guinea pigs, cows, horses, monkeys, dogs, sheep, goats, and pigs, etc. may be cited as non-human animals.

As one form of transgenic non-human animal, the present invention provides a transgenic non-human animal that transiently expresses constitutively active PKD1 in cardiomyocytes. The transgenic non-human animals in question develop cardiac hypertrophy based on the PKD1 in the cardiomyocytes being in the fully active state (phosphorylated). For this reason, as animal models having specific characteristics that resemble a human disease condition of cardiac hypertrophy (non-human animal models of cardiac hypertrophy disease), the transgenic non-human animals may be effectively used in histological research on cardiac hypertrophy, in demonstrating the mechanisms of the development into heart disease, and, as animals for screening, in development of cardiac hypertrophy suppressants and preventatives and remedies for heart disease.

Constitutively active PKD1 can be created by deleting the amino acid residue of a specific region of PKD1 or by substituting by point mutation, etc. Concretely, with human derived PKD1, constitutively active PKD1 may be created by deleting the a PH domain in the amino acid sequence thereof, or by substituting the serines of positions 744 and 748 with glutamic acids; and with mouse derived PKD1, by substituting the serines of positions 744 and 748 with glutamic acids in the amino acid sequence thereof.

The animal model of cardiac hypertrophy disease is, concretely, a transgenic non-human animal that is in a state capable of expressing the genes that code for constitutively active PKD1 in cardiomyocytes under the control of functional DNA such as a promoter, and that based on this the constitutively active PKD1 is expressed and produced in cardiomyocytes.

In another form, the present invention provides a transgenic non-human animal that has the functional expression of PKD1 in cardiomyocytes knocked out. In the transgenic non-human animals, the cardiac hypertrophy signal pathways through GPCR or EGF receptor is blocked because the PKD1 in the cardiomyocytes is in the inactive state (non-phosphorylated), and these animals do not develop cardiac hypertrophy by stimulation mediated through the GPCR or EGF receptor. For this reason, as animal models with a blocked cardiac hypertrophy signal cascade mediated through GPCR or EGF receptor, the transgenic non-human animals may be used effectively in understanding the cardiac hypertrophy mechanisms through other cardiac hypertrophy signal pathways than cardiac hypertrophy signal pathways mediated through GPCR or EGF receptor (for example, cardiac hypertrophy signal pathways mediated through gp130 receptor), and in understanding the development into heart disease.

The transgenic non-human animals with the functional expression of PKD1 in cardiomyocytes knocked out can be created by expressing dominant negative PKD1 in the cardiomyocytes thereof. Dominant negative PKD1 may be created either by deleting the amino acid residue of a specified region of PKD1 (for example, the ATP binding region or the phosphorylation active loop), or by using point mutation to substitute a specified site (for example, the ATP binding site or the phosphorylation active loop site). Concretely, with human derived PKD1, the dominant negative PKD1 may be created by substituting the lysine at position 612 in the amino acid sequence thereof with tryptophan, substituting the lysine of position 618 with asparagine, substituting the aspartic acid of position 733 with alanine, or substituting the serines of positions 738 and 742 with alanines. With mouse derived PKD1, the dominant negative PKD1 may be created by substituting the lysine of position 618 in the amino acid sequence thereof with methionine, or substituting the serines of positions 744 and 748 with alanines.

The transgenic non-human animals in question are, concretely, animals that are in a state of being able to express genes that code for dominant negative PKD1 under the control of functional DNA such as a promoter in cardiomyocytes, and thereby the dominant negative PKD1 is expressed and produced in the cardiomyocytes.

The method for producing these transgenic non-human animals is generally described in U.S. Pat. No. 4,873,191, Brister et al., Proc. Natl. Acad. Sci. USA, 82, p. 4438-4442 (1985), Japanese Laid-open Patent No. 2003-55266, and “Manipulating the Mouse Embryo: A Laboratory Manual”, edition 2 (editors Hogan, Beddington, Costantimi, and Long, Cold Spring Harbor Laboratory Press. 1994), and the present invention may be carried out in conformity to these. The contents of this literature are incorporated hereto by reference. The method of using microinjection to insert into a zygote of the test animal the desired gene to be introduced into that animal may be cited as an example of a typical method. The microinjected zygote is transplanted into a female host, and the desired transgenic non-human animal may be selected from the offspring by indexing the expression of the introduced gene.

V. Screening Method

The present invention provides a method for screening substances that can suppress the functional expression of PKD1 in cardiomyocytes. Specifically, a preferable substance is one that has an action to suppress the functional expression of PKD1 in cardiomyocytes. The substance may be expected to be able to suppress the onset or development of cardiac hypertrophy by blocking or suppressing hypertrophy signal transduction mediated through GCPR in cardiomyocytes. Further, according to the substance, it may be expected that heart disease caused by cardiac hypertrophy can be prevented or remedied. That is, the present invention provides a method for screening the active ingredients of cardiac hypertrophy suppressants, or agents to prevent or remedy heart disease caused by cardiac hypertrophy.

The screening method in question basically comprises investigating substances that have an action to suppress functional expression of PKD1 in cardiomyocytes, but concretely, the following screening methods (1) to (4) may be cited as examples.

(1) Method for screening an active ingredient of cardiac hypertrophy suppressants including the following steps:

(a) bringing a test substance into contact with cells that can express PKD1;

(b) measuring the levels of expression of PKD1 in the aforementioned cells, and comparing with the level of PKD1 expression in contrast cells that are not brought into contact with the test substance; and

(c) based on the comparative results of (b) above, selecting as an active ingredient of cardiac hypertrophy suppressants the test substance which, when brought into contact with cells, lowered the level of expression of PKD1 as compared to the contrast cells.

Using the steps, it is possible to obtain substances having an action to suppress the expression and production of PKD1.

The cells used here, irrespective of being intrinsic or extrinsic, are in a state capable of expressing PKD1 genes, and the derivation of the cells is not particularly limited. Preferably, these are cells in a state capable of expressing PKD1 genes derived from humans or derived from mammals other than humans. Concretely, cardiomyocytes or skeletal muscle cells derived from humans or from mammals other than humans may be cited, and cultured cells isolated and prepared from the heart or skeletal muscle may be suitably used. Tissue that is an aggregate of cells may also be included in the related category. It is also possible to use prokaryotic cells or eukaryotic cells (including insect cells) having PKD1 genes derived from humans or derived from mammals other than humans, in a state capable of expressing the PKD1 genes.

The test substances are not particularly limited, but are nucleic acids, peptides, proteins, organic compounds or inorganic compounds. Concretely, the screening may be carried out by bringing these test substances or substances containing them (for example, cell extracts, including expression products of gene libraries, etc.) into contact with the target cells. The conditions for contact between the cells and the test substance adopted when screening are not particularly limited, but it is preferable to select culture conditions that can express the desired gene without killing the cell (the same applies in the screening methods below).

Using a polynucleotide and/or a complementary polynucleotide thereof that has at least 15 bases continuous to the base sequence of the PKD1 gene as a primer or probe, it is possible to measure the level of PKD1 expression by employing a well-known method such as Northern blot, RT-PCR, in situ hybridization analysis, differential hybridization, DNA chip, or RNase protection assay. The expression level of PKD1 may also be evaluated by measuring the amount of PKD1 (protein) expressed and produced. In this case, the PKD1 produced is detected and assayed by a well-known method such as Western blot using as a marker an antibody that recognizes PKD1.

Selection of the active ingredient (candidate substance) of a cardiac hypertrophy suppressant or an agent to prevent or remedy heart disease caused by cardiac hypertrophy by the screening method may be conducted by using as an index the fact that the level of PKD1 expression in cells brought into contact with a test substance becomes lower than the level of PKD1 expression in cells that have not been brought into contact with the test substance.

As indicated in the experiments, PKD1 is a component that plays a central role in cardiac hypertrophy signal transduction mediated through GPCR or EGF receptor. Consequently, the substance that suppresses the expression and production of PKD1 selected by the aforementioned screening can prevent or suppress the cardiac hypertrophy signal cascade, and therefore can be used as an active ingredient of a cardiac hypertrophy suppressant or as an active ingredient of an agent to prevent or remedy heart disease caused by cardiac hypertrophy.

(2) Method for screening an active ingredient of cardiac hypertrophy suppressants including the following steps:

(a) bringing a PKD1 activator and a test substance into contact with cells that can express PKD1;

(b) measuring the activity of PKD1 in the aforementioned cells, and comparing with the activity of PKD1 in contrast cells that are not brought into contact with the test substance; and

(c) based on the comparative results of (b) above, selecting as an active ingredient of cardiac hypertrophy suppressants the test substance which, when brought into contact with cells, lower the activity of PKD1 as compared to the contrast cells.

Using the steps, it is possible to obtain substances that suppress the expression and production of PKD1 or that inhibit PKD1 activity.

Here, PKD1 activators may be ones that can make PKD1 fully active (phosphorylation), and typical examples include phorbol esters such as TPA (12-0-tetradecanoylphorbol 13-acetate), diacylglycerol (DG), and PKC (for example, PKCε).

The measurement of PKD1 activity may be conducted by evaluating the phosphorylating ability of PKD1 (for example, with human derived PKD1, the activity for phosphorylating position Ser-916 of the amino acid sequence). The method thereof is not particularly limited, but concretely, the method of evaluating phosphorylating ability (phosphorylation assay) for a specific substrate peptide (for example, Syntide-2 [APLARTLSVAGLPGKK]) may be cited as an example. Specifically, the phosphorylation assay in question may be conducted by following the methods explained in the experiments.

Using the screening method in question, selection of the active ingredient (candidate substance) of a cardiac hypertrophy suppressant or an agent to prevent or remedy heart disease caused by cardiac hypertrophy may be conducted by using as an index the fact that the PKD1 activity in cells brought into contact with a test substance becomes lower than the PKD1 activity in cells that have not been brought into contact with the test substance.

As indicated in the experiments, PKD1 is an important component in cardiac hypertrophy signal transduction, and a downstream cascade is activated through PKD1 activity, thereby inducing cardiac hypertrophy. Consequently, a substance selected by the aforementioned screening method can inhibit or suppress the cardiac hypertrophy signaling, and can be used as an active ingredient of a cardiac hypertrophy suppressant or as an active ingredient of an agent to prevent or remedy heart disease caused by cardiac hypertrophy.

(3) Method for screening an active ingredient of cardiac hypertrophy suppressants including the following steps:

(a) bringing a test substance and a cardiac hypertrophy inducer that stimulate GPCR or EGF receptor into contact with cardiomyocytes;

(b) measuring the PKD1 activity, localization of phosphorylated PKD1 in sarcomere Z-discs, or the intermolecular distance of PKC_{ε} and PKD1 in the aforementioned cardiomyocytes, and comparing with the corresponding PKD1 activity, localization or intermolecular distance in contrast cardiomyocytes that were brought into contact with hypercardia inducer only; and

(c) based on the comparative results of (b) above, selecting as an active ingredient of cardiac hypertrophy suppressants the test substance which, when brought into contact with the cardiomyocytes, lower the activity of PKD1, lower the localization of phosphorylated PKD1 in sarcomere Z-discs, or increase the intermolecular distance of PKC_{ε} and PKD1 as compared to the contrast cardiomyocytes.

Using the steps, it is possible to obtain substances that suppress the functional expression of PKD1 in cardiomyocytes irrespective of the mechanism of action.

Cardiomyocytes derived from humans or from mammals other than humans may be cited as the cardiomyocytes. Cultured cells suitably isolated and prepared from humans or from mammals other than humans may be used as the cells in question. Tissue that is an aggregate of cardiomyocytes may also be included in this category. Substances known to induce cardiac hypertrophy mediated through GPCR or EGF receptor may be cited as cardiac hypertrophy inducers that stimulate GPCR or EGF receptor. As examples of the substances, typical of the former are AngII, endothelin-1, and NE; and of the latter, epidermal growth factor (EGF).

In the screening method, the desired substance is selected by using as an index at least one of PKD1 activity in cardiomyocytes, localization of phosphorylated PKD1 in sarcomere Z-discs, or intermolecular distance of PKC_{ε} and PKD1.

The method described in (2) above may be used as the measurement method of PKD1 activity.

The localization of phosphorylated PKD1 in sarcomere Z-discs can be investigated by staining the cardiomyocytes with a regent that can specifically label and detect phosphorylated PKD1 (fully active PKD1), and then observing the behavior of phosphorylated PKD1 in cardiomyocytes using a detection method corresponding to that reagent. Antibodies that specifically recognize and bond with phosphorylated PKD1 may be cited as regents that can specifically label and detect phosphorylated PKD1. Specifically, with human derived PKD1, examples include antibodies to peptides having the amino acid sequence 912 to 918 (here, position Ser-916 is phosphorylated) of human derived PKD1 (anti-fully active PKD1 antibodies), and these antibodies will be used in the experiments. Preferably, the method may be cited of treating cardiomyocytes with an antibody specific to α-actinin sarcomere, an antibody to the aforementioned phosphorylated PKD1 (fully active PKD1), and a fluorescent or chemiluminescent reagent, and then conducting a differential analysis of the fluorescent image or chemiluminescent image of the cells obtained. A more concrete measurement method is described in the experiments.

The intermolecular distance of PKC_{ε} and PKD1 within the cardiomyocytes can be calculated by determining FRET (fluorescence resonance energy transfer) using fluorescent proteins. Specifically, the method can be conducted using, for example, a 3CCD-FRET imaging system (AQUACOSMOS/ASHURA) manufactured by Hamamatsu Photonics Co. (Japan), and according to the method, it is possible to quantitatively monitor the interaction of different proteins (PKC_{ε} and PKD1).

Selection of an active ingredient (candidate substance) of cardiac hypertrophy suppressants or agents to prevent or remedy heart disease caused by cardiac hypertrophy based on the screening method may be conducted by using as an index as follow:
the fact that PKD1 activity in cardiomyocytes that have been brought into contact with a cardiac hypertrophy inducer and a test substance is lower than the PKD1 activity in contrast cardiomyocytes that have been brought into contact with cardiac hypertrophy inducer only (that is, not brought into contact with the test substance);
the fact the localization of phosphorylated PKD1 in sarcomere Z-discs in cardiomyocytes that have been brought into contact with a cardiac hypertrophy inducer and a test substance is lower than the localization of phosphorylated PKD1 in sarcomere Z-discs in contrast cardiomyocytes that have been brought into contact with cardiac hypertrophy inducer only; or
the fact that the intermolecular distance of PKC_{ε} and PKD1 in cardiomyocytes that have been brought into contact with a cardiac hypertrophy inducer and a test substance is longer than the intermolecular distance of PKC_{ε} and PKD1 in contrast cardiomyocytes that have been brought into contact with cardiac hypertrophy inducer only.

As indicated in the experiments, PKD1 is an important component in cardiac hypertrophy signal transduction, and a downstream cascade is activated through PKD1 activation, thereby inducing cardiac hypertrophy. In addition, when activated, PKD1 moves to sarcomere Z-discs and becomes localized there. Further, PKC_{ε} is a direct activator of PKD1, and during cardiac hypertrophy signaling, PKC_{ε} and PKD1 form a complex. Consequently, a substance selected by the aforementioned screening methods has an action to prevent or suppress cardiac hypertrophy signaling, and can be used as an active ingredient of a cardiac hypertrophy suppressant or as an active ingredient of an agent to prevent or remedy heart disease caused by cardiac hypertrophy.

(4) Method for screening an active ingredient of cardiac hypertrophy suppressants including the following steps:

(a) bringing a test substance into contact with cardiomyocytes that can express constitutively active PKC_{ε} or constitutively active PKD1;

(b) measuring the PKD1 activity, localization of phosphorylated PKD1 (fully active PKD1) in sarcomere Z-discs, or the intermolecular distance of PKC_{ε} and PKD1 in the aforementioned cardiomyocytes, and comparing with the PKD1 activity, localization or intermolecular distance in corresponding contrast cardiomyocytes that were not brought into contact with the test substance; and

(c) based on the comparative results of (b) above, selecting as an active ingredient of cardiac hypertrophy suppressants the test substances which, when brought into contact with the cardiomyocytes, lower the activity of PKD1, lower the localization of phosphorylated PKD1 in sarcomere Z-discs, or increase the intermolecular distance of PKC_{ε} and PKD1 compared to the contrast cardiomyocytes.

Using the steps, it is possible to obtain substances that suppress the activation of PKD1 in cardiomyocytes.

Here, as the cardiomyocytes, cardiomyocytes prepared to be able to express constitutively active PKC_{ε} or constitutively active PKD1 are used. Cardiomyocytes that can express constitutively active PKD1 are able to express PKD1 in a fully active state (phosphorylated) without stimulation by cardiac hypertrophy inducers. Cardiomyocytes that can express constitutively active PKC_{ε} are able to fully activate (phosphorylate) PKD1 based on activation of PKCε without stimulation by cardiac hypertrophy inducers.

For example, constitutively active PKC_{ε} has the psuedosubstrate region of the N-terminal of PKC_{ε} deleted, or has a specified amino acid residue substituted by point mutation (Schönwasser, D. C., et al., Mol. Cell. Biol. 18, p. 790-798, 1998). In the case of human derived PKC_{ε}, protein with deleted region 156 to 162 in the amino acid sequence of human derived PKC_{ε} may be cited as a concrete example.

For example, constitutively active PKD1 has the amino acid residue of a specific region of PKD1 deleted or substituted by a point mutation, etc. In the case of human derived PKD1, deletion of the PH domain in the amino acid sequence thereof, or substitution of the serines in positions 744 and 748 with glutamic acids in the amino acid sequence thereof may be cited as concrete examples. In mouse derived PKD1, substitution of the serines of positions 744 and 748 with glutamic acids in the amino acid sequence thereof may be cited as an example.

The cardiomyocytes that can express the constitutively active PKC_{ε} and the constitutively active PKD1 can be created by introducing genes that code for these proteins into cardiomyocytes (including culture cells) derived from humans or other mammals following ordinary genetic recombinant technologies.

Selection of the active ingredient (candidate substance) of a cardiac hypertrophy suppressant or an agent to prevent or remedy heart disease caused by cardiac hypertrophy based on the screening method in question may be conducted by using as an index as follows:
the fact that PKD1 activity in cardiomyocytes that have been brought into contact with a test substance is lower than the PKD1 activity in contrast cardiomyocytes that have not been brought into contact with the test substance;
the fact that the localization of PKD1 in sarcomere Z-discs in cardiomyocytes that have been brought into contact with a test substance is lower than the localization of PKD1 in sarcomere Z-discs in contrast cardiomyocytes that have not been brought into contact with the test substance; or
the fact that the intermolecular distance of PKC_{ε} and PKD1 in cardiomyocytes that have been brought into contact with a test substance is longer than the intermolecular distance of PKC_{ε} and PKD1 in contrast cardiomyocytes that have not been brought into contact with the test substance.

The substances selected by aforementioned screening method have an action to prevent or suppress cardiac hypertrophy signaling, and may be used as an active ingredient of a cardiac hypertrophy suppressant or as an active ingredient of an agent to prevent or remedy heart diseases caused by cardiac hypertrophy.

Candidate substances selected by the above screening methods may also be screened using non-human animal models of cardiac hypertrophy or of diseases caused cardiac hypertrophy.

The previously described transgenic non-human animals of the present invention that transiently expressed constitutively active PKD1 may be cited as examples of the said non-human animal models. The screening using the non-human animal models may be carried out according to the following steps:

(a) administering a test substance to a transgenic non-human animal (non-human animal model of cardiac hypertrophy or of disease caused by cardiac hypertrophy) with transient expression of constitutively active PKD1;

(b) measuring the extent of cardiac hypertrophy in the aforementioned non-human animal, and comparing with the extent of cardiac hypertrophy in contrast transgenic non-human animal that were not administered the test substance; and

(c) based on the comparative results of (b) above, selecting test substance that reduced or suppressed cardiac hypertrophy of the non-human animal as cardiac hypertrophy suppressants.

The extent of cardiac hypertrophy may be determined by histological evaluation of the heart, clinical evaluation (echocardiogram, Doppler ultrasound exam, coronary artery imaging, chest X-ray, electrocardiogram, etc.), or by evaluation from the level of ANP expression in cardiomyocytes, which is a marker of cardiac hypertrophy.

Once a candidate substance has been selected, pharmacological tests using non-human animals with cardiac hypertrophy or diseases caused by cardiac hypertrophy, safety tests, and clinical trials on patients (human) with cardiac hypertrophy or diseases caused by cardiac hypertrophy, or patients (human) with the preconditions of cardiac hypertrophy may also be conducted; and by conducting these tests, it is possible to select a more practical active ingredient for a composition for suppressing cardiac hypertrophy or for an agent to prevent or remedy heart disease caused by cardiac hypertrophy.

After constitutively analyzing as necessary, substances selected in this way may be industrially manufactured by chemical synthesis, biological synthesis (including fermentation) or genetic manipulation corresponding to the type of substance, and then used in the preparation of pharmaceutical compositions to suppress cardiac hypertrophy or pharmaceutical compositions to prevent or remedy heart disease.

### EXPERIMENTS

The present invention will be described below in further detail based on experiment, but the present invention is not limited to any of the following experiments. The genetic engineering technology and molecular-biological experimental techniques used in the present invention are common, widely used methods, and may be conducted by following the methods described, for example, in J., Sambrook, E. F., Frisch, T., Maniatis: Molecular Cloning 2nd edition, published by Cold Spring Harbor Laboratory Press, 1989; and D. M. Glover: DNA Cloning, published by IRL, 1985; etc.

(1) Abbreviations

Unless specifically stated, the abbreviation used in the following experiments shall mean the following terms.

AngII: Angiotensin II

ANF: Arterial natriuretic factor

BrDU: 5-bromo-2′-deoxyuridine

BSA: Bovine serum albumin

CA Constitutively active

DN: Dominant negative

FBS: Fetal bovine serum

GFP: Green fluorescent protein

G protein: Guanine nucleotide-binding protein

GPCR Seven transmembrane-spanning heterotrimeric G protein-coupled receptors

KD: Kinase dead

LIF: Leukemia inhibitory factor

NE: Norepinephrine

ET1 Endothelin 1

EGF: Epidermal growth factor

bFGF: Basic fibroblast growth factor

NRC: Neonatal rat cardiomyocytes

PBS: Phosphate-buffered saline

PKC: Protein kinase C

PKD: Protein kinase D

PLC: Phospholipase C

TPA: 12-0-tetradecanoylphorbol 13-acetate)

DMEM: Dulbecco's modified Eagles Medium

(2) Materials

The basic materials used in the following experiments are as follows:

(i) Lysis buffer solution A: 50 mM Tris (pH 7.4), 150 mM NaCl, 1.3% Triton X-100, 1 mM EDTA, 1 mM dithiothreitol, 50 mM NaF, 1 mM Na₃VO₄, and protease inhibitor cocktail tablet (manufactured by Roche) 1 tablet (per 50 mL)

(ii) Antibodies:

\textcircled{1} Anti-PKD1/2 monoclonal antibody (manufactured by LC Laboratories): it has reactivity to both PKD1 and PKD,

\textcircled{2} anti-sarcomeric-α-actinin monoclonal antibody (clone EA-53: manufactured by SIGMA),

\textcircled{3} anti-H3 histone mouse monoclonal antibody (05-499: manufactured UpState Biotechnology),

\textcircled{4} anti-fully active PKD1 polyclonal antibody: it was manufactured from blood of rabbit inoculated with peptides in which the C-terminal region (Ser-916) of human derived PKD1 was phosphorylated (amino acid region 912 to 918 of human derived PKD1: SERVPSIL),

\textcircled{5} anti-inactive PKD1 polyclonal antibody: it was manufactured from blood of rabbit inoculated with peptides of the C-terminal region of human derived PKD1 (amino acid region 904 to 918 of human derived PKD1: EEREMKALSERVSIL).

The human derived PKD1 is initially made catalytically active by phosphorylation of the active loop residues (Ser-744, Ser-748) using phosphoinositide-dependent kinase 1 (PDK1). Then the C-terminal region (Ser-916) undergoes auto-phosphorylation, and the entire molecule becomes the active type (in the present description, this is called the “fully active type”). Accordingly, phosphorylation of Ser-916 site of PKD1 provides the optimum indication to confirm that PKD1 is activated in vivo (fully active PKD1) (Matthews S. A., et al., J. Biol. Chem. 274, p. 26543-26549).

Consequently, the antibodies to the phosphorylated Ser-916 site (concretely, antibodies to PKD1 fragments [peptide fragments] which have a phosphorylated Ser-916 site: \textcircled{4} anti-fully active PKD1 polyclonal antibody), which are used in the following experiments, are antibodies effective for specifically detecting fully active PKD1 present in cardiomyocytes.

(3) pTB701-HA Vector

A vector for expressing fused protein with HA epitope at the NH₂ terminal in mammal cells. The base sequence that codes for HA epitope is inserted into downstream of the SV40 early promoter in the expression vector pTB701.

(4) Constitutively Active PKC Mutant (CA-PKC)

This is in fully active state either by deleting the pseudo-substrate region of the NH₂ terminal or by point mutation. Acquired from Prof. Parker (Schönwasser, D. C., et al., Mol. Cell. Biol. 18, p. 790-798, 1998).

\textcircled{1} CA-PKCβI: Region 22-28 of the amino acid of human derived PKCβI is deleted

\textcircled{2} CA-PKCδ: Ala of position 147 of the amino acid of human derived PKCδ is substituted with Glu

Both of these are expressed using pCO2 vector.

\textcircled{3} CA-PKCε: Region 156-162 of the amino acid of human derived PKCε is deleted

This is expressed using pMT2 vector.

(5) Dominant Negative PKC Mutant (DN-PKCs)

This is in the inactive state by mutating (point mutation) of Lys at the ATP binding site into Met (Kuroda, S., et al., J. Biol. Chem. 271, p. 31029-31032, 1996; Kuroda, S., et al., J. Cell Biol. 144, p. 403-411, 1999).

\textcircled{1} PKCβI-HA dominant negative mutant (K371M PKCβI-HA)

Prepared using site-directed mutagenesis to substitute Met for the Lys necessary for ATP binding at position 371 of rat derived PKCβI.

\textcircled{2} PKCξ-HA dominant negative mutant (K281M PKCξ-HA)

Prepared using site-directed mutagenesis to substitute Met for the Lys necessary for ATP binding at position 281 of rat derived PKCξ.

\textcircled{3} PKCε-HA dominant negative mutant (K440M PKCε-HA)

Prepared using site-directed mutagenesis to substitute Met for the Lys necessary for ATP binding at position 440 of rat derived PKCε.

(6) Constitutively Active PKD1 (CA-PKD1)

Prepared by substituting the Ser of amino acid positions 744 and 748 of human derived PKD1 with Glu respectively, and this is the fully active state based on this mutation (S744E/S748E PKD1) (pEGFPC1-PKD-CA).

(7) Dominant Negative PKD1 (DN-PKD1)

Prepared by substituting the Lys of amino acid position 618 of human derived PKD1 with Asn, and this is the inactive state based on this mutation (SK618N PKD1) (pEGFPC1-PKD-DN).

### Experiment 1

Expression of PKD1 in Neonatal Rat Cardiomyocytes and Localization thereof

Western blotting is used to investigate the state of expression of PKD1 and PKD2 in neonatal rat cardiomyocytes (NRC), as well as the intracellular localization (distribution) thereof. PKD2 is a gene product that differs from PKD1 (Sturany, S., et al., J. Biol. Chem. 276, p. 3310-3318, 2001). NRC was further treated with TPA, and the expression of PKD1 and PKD2 in the treated NRC and the intracellular localization thereof were studied in the same manner.

TPA is one type of phorbol ester known to be an activator of PKC and PKD (Kikkawa U., et al., Adv. Cyclic Nucleotide Protein Phosphorylation Res., 17, p. 437-42, 1984; Valverde AM, et al., Proc. Natl. Acad. Sci. USA, 30, 91(18), p. 8572-6, 1994).

TPA also induces cardiac hypertrophy (Kinnunen P., et al., Br. J. Pharmacol., 102(2), p. 453-61, 1991), but the inducement of cardiac hypertrophy thereby is ultimately based on activation of PKC, and it was not at all known that PKD mediates in inducing cardiac hypertrophy.

(1) Isolation of NRC and Primary Culture thereof

Hearts removed from neonate rats within one month of birth were cultured using a method similar to the method of Goshima et al. (Goshima K., et al., J. Mol. Cell Cardiol., 1977 Jan. 9(1), p. 7-23).

Concretely, the hearts were removed from 30 neonate rats, then rinsed of blood with phosphate-buffered physiological saline (PBS, containing no Mg²⁺ or Ca²⁺), removed the blood vessels, divided into 4 pieces, and then rinsed again with PBS. Next, 0.3 g of the heart tissue was processed in 10 mL of 0.1 w/v % collagenase type I (Wako Pure Chemical Industries Ltd.) aqueous solution for 10 to 15 minutes at 37° C., and the cardiomyocytes were separated and dispersed in the aqueous solution. This collagenase treatment was repeated 2 more times using fresh collagenase aqueous solution. The treatment solution was centrifugally separated, the resultant precipitate were suspended in 10 mL of culture medium (DMEM+10% FBS), and the suspension was filtrated with sterilized Kimwipes set in a sterile filter unit (manufactured by Millipore) to isolate Cardiomyocytes (dispersed cells).

The cardiomyocytes obtained were moved to a tissue culture plate (manufactured by Falcon) coated with collagen, and cultured for 50 minutes, at 37° C. under a 5% CO₂ concentration, and utilizing the difference in the adhesive strength of the cells, the fibroblast cells co-present in the cardiomyocytes were removed by adhering to the bottom of a culture plate coated with collagen (a differential adhesion technique). The cardiomyocytes present in the supernatant were used as neonatal rat cardiomyocytes (NRC).

The NRC obtained was stored in DMEM containing 0.45% glucose, 10% (v/v) fetal bovine serum (FBS), 2 mM L-glutamine, 100 units/mL penicillin, 100 μg/mL streptomycin, and 20 μM 5-bromo-2′-deoxyuridine (BrDU: Sigma).

(2) TPA Processing, and Cell Fractionation

The NRC prepared above was moved to non-serum DMEM (manufactured by Nacalai Tesque), and cultured overnight (5% CO₂ concentration, 37° C.). Then, TPA was added to the culture to make a final concentration of 100 nM, and incubated for 20 minutes. Next, the cardiomyocytes were recovered and rinsed in PBS, subjected to bacteriolysis by treating with Lysis buffer solution (having the same composition as Lysis buffer solution A except that the Triton X-100 was 0.1%), centrifuged (15000 rpm, approximately 10 minutes), and fractioned into supernatant (cytoplasm fraction) and precipitate. The precipitate was further dissolved in Lysis buffer solution A. This was lightly separated by centrifuge, and the supernatant obtained was taken as the cell membrane fraction. As a control, NRC that was not treated with TPA was subjected to bacteriolysis, and the cytoplasm and cell membrane fractions were separated in the same manner as above (untreated substance).

(3) Western Blotting

The TPA treated substance (cytoplasm fraction and cell membrane fraction) and the untreated substance (cytoplasm fraction and cell membrane fraction) prepared as above were subjected to 10% SDS-polyacrylamide gel electrophoresis (SDS-PAGE) so that the same amount of protein was added per lane respectively. After electrophoretic migration, this was transferred to polyvinylidene difluoride (PVDF) membrane (manufactured by Immobilon-P, Millipore). Next, Western blotting was conducted using the following antibodies: \textcircled{1} anti-PKD1/2 monoclonal antibody (manufactured by LC Laboratories), \textcircled{2} anti-fully active PKD1 polyclonal antibody, \textcircled{3} anti-inactive PKD1 polyclonal antibody, \textcircled{4} anti-sarcomeric-α-actinin monoclonal antibody (clone EA-53: manufactured by SIGMA), and \textcircled{5} anti-H3 histon mouse monoclonal antibody (05-499: manufactured by UpState Biotechnology). The PKD1, PKD2, fully active PKD1, inactive PKD1, α-actinin, and histone H3 contained in the various samples (TPA treated substance (cytoplasm fraction and cell membrane fraction) and the untreated substance (cytoplasm fraction and cell membrane fraction) were stained.

Next, anti-mouse or anti-rabbit IgG bound with horseradish derived peroxidase (manufactured by Amersham Pharmacia) was used as the second antibody reagent, so that it is possible to observe the blotting results with chemical luminescence on X-ray film.

(4) Results

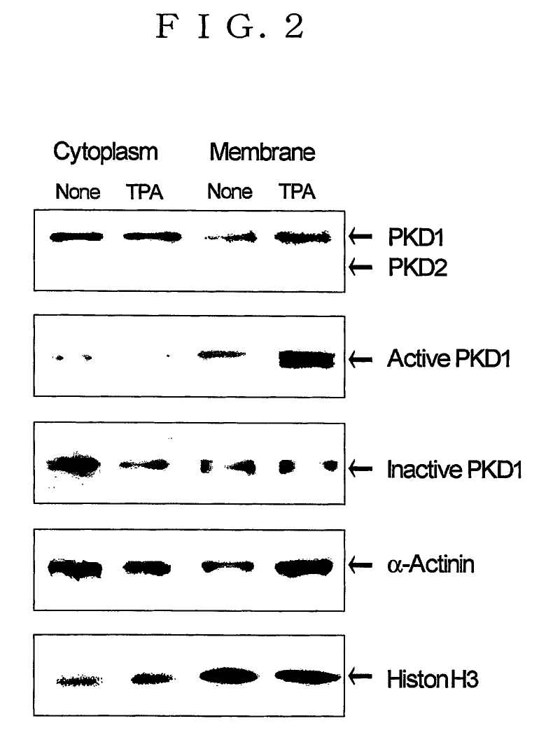

The results are indicated in FIG. 2. Nearly the same amount of α-actinin and histone H3 was detected in the various samples (cytoplasm fraction: untreated substance (None) and TPA treated substance (TPA), and membrane fraction: untreated substance (None) and TPA treated substance (TPA)). This fact means that nearly equivalent amounts of cardiomyocytes derived protein were present in every lane.

The results of FIG. 2 demonstrate that only PKD1 was present in cardiomyocytes, and that PKD2 was not present. (The present inventors have already used the same method to confirm that PKD3 is not present in cardiomyocytes.) Fully active PKD1 was present in large amounts in the membrane fraction of the cardiomyocytes treated with PKD or TPA, which is PKC activator, and hardly any was expressed either of in the cytoplasm fraction of the TPA-treated NRC or in the fractions of TPA-untreated NRC (refer to the second column from the top in FIG. 2). This fact indicates that TPA treatment of cardiomyocytes causes phosphorylation of the Ser-916 site of PKD1 in cardiomyocytes to make the PKD1 fully active; and fully activated PKD1 (phosphorylated PKD1) becomes to have high affinity with cell membrane or proteins in cell membrane fraction by changing the structure, and moves from cytoplasm to cell membrane (localization into membrane).

### Experiment 2

Behavior of Fully Active PKD1 in Cardiomyocytes

NRC prepared by a method similar to the method as described in (1) of Experiment 1 was moved to a glass culture plate (35 mm diameter) coated with poly-L-lysine, and was cultured over night (5% CO₂ concentration, 37° C.) in DMEM without blood serum (manufactured by Nacalai Tesque). TPA was added to this to make a final concentration of 20 nM, and this was cultured a further 18 hours (5% CO₂ concentration, 37° C.) (approximately 1×10³ cells). After culturing, the cells obtained were rinsed 2 times with PBS, fixed by treating with 4 w/v % paraformaldehide for 30 minutes at room temperature, treated by soaking in PBS containing 0.25 v/v % Triton X-100 for 30 minutes at 4° C., and then incubated in blocking buffer solution (PBS containing 3 w/v % BSA, 2 v/v % FBS, 1 v/v % normal goat blood serum, and 0.03 v/v % Triton X-100).

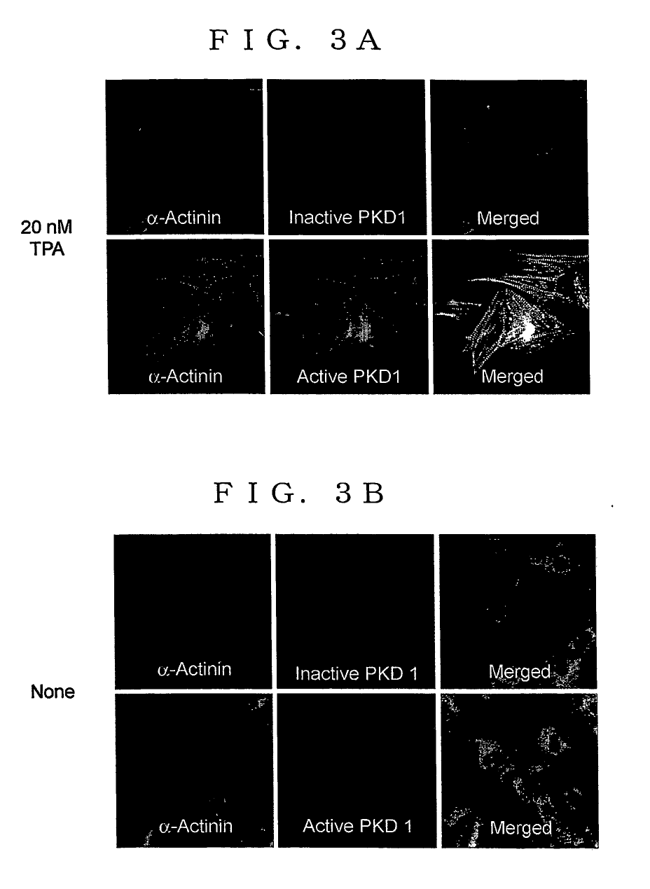

The cells obtained were incubated at 4° C. for 16 hours in blocking buffer solution containing antibodies (anti-fully active PKD1 polyclonal antibody, anti-inactive PKD1 polyclonal antibody, and anti-sarcomeric-α-actinin monoclonal antibody). This was rinsed 2 times in PBS, moved into PBS containing 0.3 v/v % Cy3-labeled anti-mouse IgG antibody, and 0.3 v/v % Cy2-labeled anti-rabbit IgG antibody (second antibody) (both manufactured by Amersham Pharmacia), and incubated for 1 hour at room temperature. The fluorescent signals of the cells were observed by a confocal laser scanning microscope LSM5 Pascal (manufactured by Carl Zeiss Inc.). The results are indicated in FIG. 3A. Untreated NRC (None), which was not treated with TPA, was labeled with antibodies in the same manner as a contrast, and the results of observing the cell luminescence signals by confocal laser microscope (LSM5 Pascal) are indicated in FIG. 3B.

The α-actinin in the cardiomyocytes bound with the anti-sarcomeric-α-actinin monoclonal antibody and the second antibody (fluorescent color label antibody), and is observed as the red fluorescent signal image (in the left column in FIG. 3A, the red signals indicate α-actinin in the cardiomyocytes). As is clear by comparing FIGS. 3A and 3B, sarcomere structures were formed in TPA-treated cardiomyocytes (visualization of Z-discs), confirming that cardiac hypertrophy was induced.

The upper middle image of FIG. 3A was obtained by treating cardiomyocytes with anti-inactive PKD1 polyclonal antibody and a second antibody (fluorescent color labeling antibody), and the lower middle image of FIG. 3A was obtained by treating with anti-fully active PKD1 polyclonal antibody and a second antibody (fluorescent color labeling antibody). Specifically, the upper middle image indicates the intracellular localization of inactive PKD1, and the lower middle image indicates the intracellular localization of fully active PKD1. These results indicate that in contrast to the inactive PKD1 remaining in the nuclear vicinity of the cardiomyocytes, fully active PKD1 exhibits an image the same as that of α-actinin (comparison with the lower left image of FIG. 3A), and therefore demonstrate that fully active PKD1 concentrates in the vicinity of sarcomere structure Z-discs of cardiomyocytes. In the right column of FIG. 3A are merged images of the images in the left and middle columns, and the more yellow (red×green) presented, the more agreement between both images.

These results demonstrate that, by treating cardiomyocytes with TPA, cardiac hypertrophy is induced, PKD1 comes into a fully active state, and the fully activated PKD1 moves to the sarcomere Z-disc region. It appears that normally proteins present in sarcomere Z-discs are related to cardiac hypertrophy, and therefore, the above results suggest that PKD1 is related to cardiac hypertrophy signal transduction.

### Experiment 3

Fully Active PKD1 Behavior in Cardiomyocytes through Inducing Cardiac Hypertrophy

NE (norepinephrine), AngII (angiotensin II), and LIF (leukemia inhibition factor) are well known as substances that cause cardiac hypertrophy.(Fischer J E., et al., Nature. 207, p. 951-953, 1965; Sanchez Torres G., et al., Arch. Inst. Cardiol. Mex., 48(3), p. 549-561, 1978; Matsui H., et al., Res. Commun. Mol. Pathol. Pharmacol. 93, p. 149-162, 1996). It is also known that of the aforementioned cardiac hypertrophy inducers, NE and AngII activate PKC, and that PKCε, which is a PKC subunit, is specifically activated by AngII and moves to Z-discs (Disatnik M H, et al., Exp. Cell Res., 1994 February, 210(2), p. 287-97). It is further known that the expression and activity of PKD1 in cardiomyocytes is controlled by NE (Fischer J E., et al., Nature, 1965 Aug. 28, 207, p. 951-953; Haworth, R. S., et al., J. Mol. Cell Cardiol. 2000, 32, p. 1013-1023).

We treated NRC with various types of cardiac hypertrophy inducers (NE, AngII, LIF), and used the same method as in Experiment 2 to study the distribution (localization) of fully active PKD1 in said cardiomyocytes. NE is a cardiac hypertrophy inducer that acts on α-adrenaline-like receptors, and in the experiment below, NE was used along with propranol to block β-adrenergic receptor activity.

Specifically, NRC prepared by the same method as described above in (1) of Experiment 1 was moved to a glass culture plate (35 mm diameter) coated with poly-L-lysin, and was cultured over night (5% CO₂ concentration, 37° C.) in DMEM without blood serum (manufactured by Nacalai Tesque). Five samples of the culture cells in question were used and added respectively to \textcircled{1} 100 μM (final concentration, same hereinafter) NE (containing 2 μM propranol), \textcircled{2} 100 μM AngII, \textcircled{3} 20 nM LIF, \textcircled{4} 100 μM NE (containing 2 μM propranol)+400 nM GF109203X (concretely, treated for 30 minutes by adding GF109203X, and then NE was added), \textcircled{5} 20 nM LIF+400 nM GF109203X (concretely, treated for 30 minutes by adding GF109203X, and then LIF was added), and these were cultured a further 38 hours (5% CO₂ concentration, 37° C.) (approximately 1×10³ cells). Here, GF109203X is a selective PKC inhibitor that does not act on PKD (Zugaza J. L., et al., The EMBO J., 1996, 15, p. 6220-6230).

After culturing, the cells were rinsed 2 times with PBS, fixed by treating with 4 w/v % paraformaldehide for 30 minutes at room temperature, treated by soaking in PBS containing 0.25 v/v % Triton X-100 for 30 minutes at 4° C., and then incubated in blocking buffer solution (PBS containing 3 w/v % BSA, 2 v/v % FBS, 1 v/v % normal goat blood serum, and 0.03 v/v % Triton X-100). The cells obtained were incubated at 4° C. for 16 hours in blocking buffer solution containing antibodies (anti-fully active PKD1 polyclonal antibody, anti-inactive PKD1 polyclonal antibody, and anti-sarcomeric-α-actinin monoclonal antibody). This was rinsed 2 times in PBS, moved into PBS containing 0.3 v/v % Cy3-labeled anti-mouse IgG antibody, and 0.3 v/v % Cy2-labeled anti-rabbit IgG antibody (second antibody) (both manufactured by Amersham Pharmacia), and incubated for 1 hour at room temperature. The fluorescent signals of the cells were observed by a confocal laser scanning microscope LSM5 Pascal (manufactured by Carl Zeiss Ltd.).

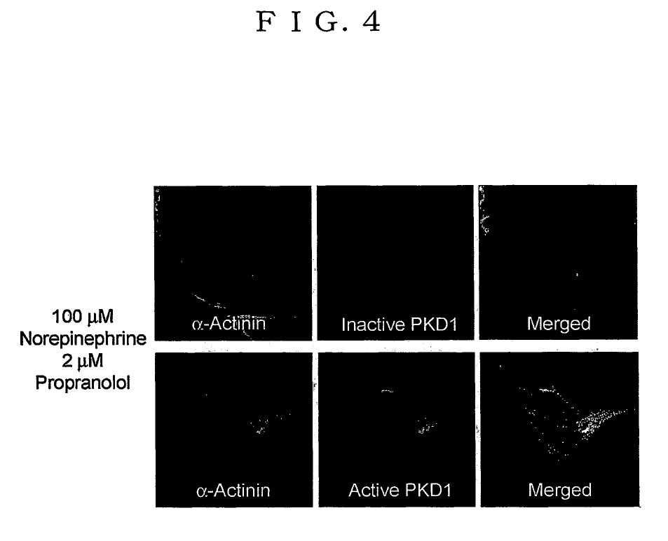

The results obtained for cells with \textcircled{1} NE treatment, \textcircled{2} AngII treatment, \textcircled{3} LIF treatment, \textcircled{4} NE+GF109203X treatment, and \textcircled{5} LIF+GF109203X treatment are indicated in the upper and lower rows of FIGS. 4, 5, 6, 7A and 7B respectively.

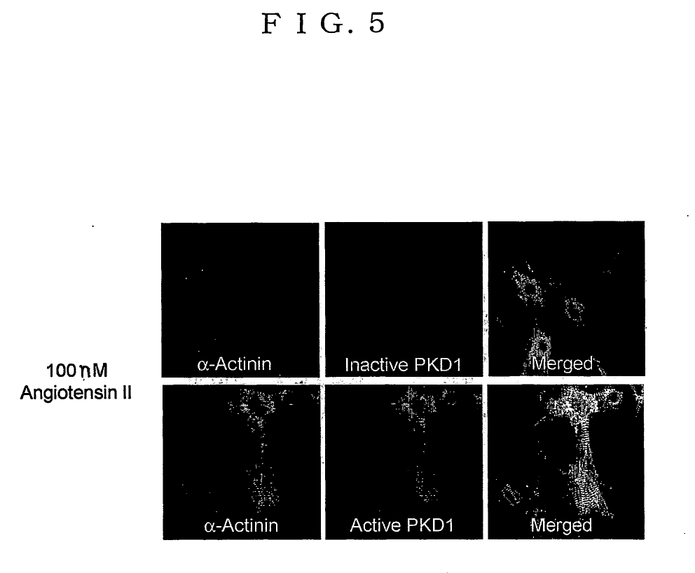

As demonstrated in the results of FIGS. 4 and 5, when treating cardiomyocytes with \textcircled{1} NE or \textcircled{2} AngII, which are cardiac hypertrophy inducers, the cells of both formed sarcomere structures (visualization of Z-discs) and induced cardiac hypertrophy. Moreover, in both case, PKD1 was fully activated (phosphorylated), and its movement to the vicinity of the sarcomere Z-discs in the cardiomyocytes was observed. However, the inactive PKD1 remained in the nuclear vicinity of the cardiomyocytes in both \textcircled{1} NE and \textcircled{2} AngII cases.

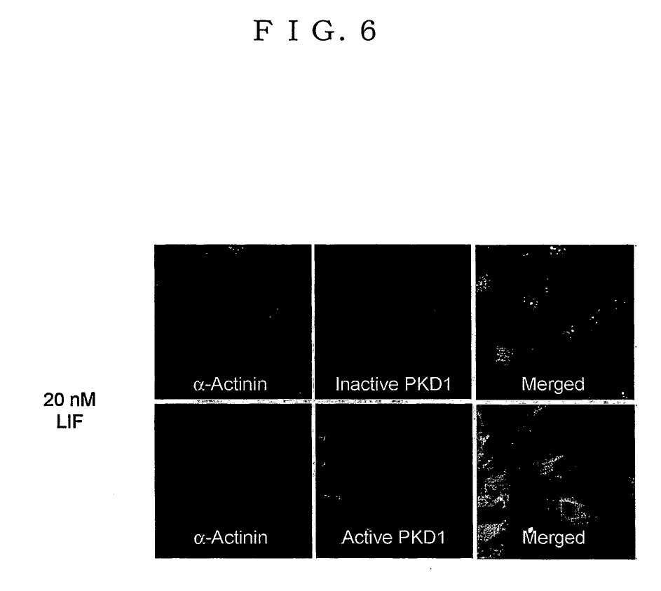

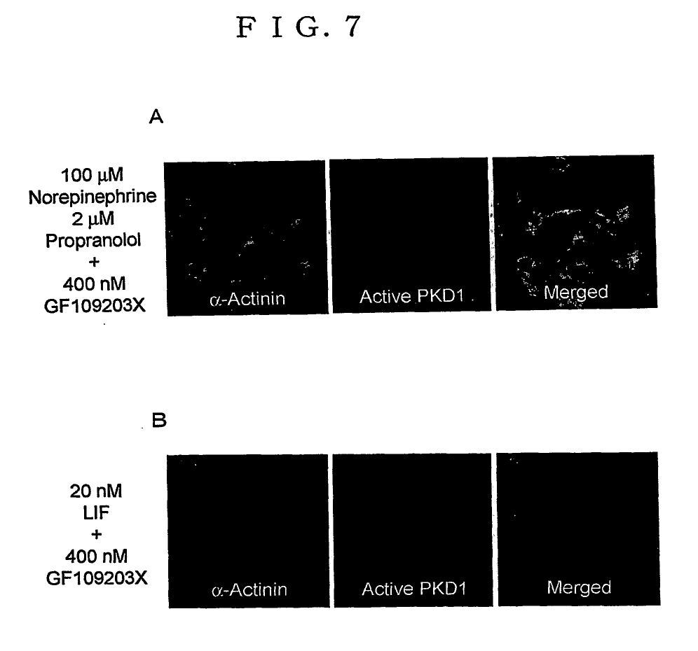

Meanwhile, as indicated in FIG. 6, when treating cardiomyocytes with \textcircled{3} LIF, which is cardiac hypertrophy inducer the same as NE and AngII, in spite of forming sarcomere structures (visualization of Z-discs) and inducing cardiac hypertrophy, no full activation (phosphorylation) of PKD1 and no movement of PKD1 to the vicinity of the sarcomere Z-discs was revealed. As shown in FIG. 7A, when inhibiting PKC activation. by pre-treating cardiomyocytes with GF109203X, no cardiac hypertrophy is generated even if treating with cardiac hypertrophy inducers (NE), and no fully activated PKD1 (phosphorylation) and no movement to sarcomere Z-discs were observed.

It is not indicated here, but when allowing endothelin 1 (ET1) and epidermal growth factor (EGF), which are known to have a cardiac hypertrophy action, to act in cardiomyocytes, the same experimental results were obtained as those indicated in FIGS. 4 and 5 for NE and AngII, respectively. Meanwhile, when allowing basic fibroblast growth factor (bFGF), which is known to have a cardiac hypertrophy action, to act on cardiomyocytes, the same results as those indicated in FIG. 6 for LIF were obtained.

The above results demonstrate that: (a) there are multiple signaling pathways that generate cardiac hypertrophy such as a cardiac hypertrophy signaling pathway that is induced by NE, AngII, ET1, and EGF, and a cardiac hypertrophy signaling pathway that is induced by LIF and bFGF, and PKD1 is a component related to cardiac hypertrophy signal transduction induced by the former cardiac hypertrophy inducers; (b) PKD1 is fully activated (phosphorylated) through the cardiac hypertrophy signal transduction induced by the former cardiac hypertrophy inducers, and migrates to the sarcomere Z-disc region of the cardiomyocytes; and (c) the full activation (phosphorylation) of PKD1 in the cardiac hypertrophy signaling pathway in question and the movement thereof to Z-discs is generated through activation of PKC.

In contrast to the fact that cardiac hypertrophy induced by NE, AngII, and ET1 appears to be generated through activation of G protein mediated through seven transmembrane-spanning heterotrimeric receptors, the mechanism of generating cardiac hypertrophy based on LIF is mediated through gp130 receptors. Specifically, the above results indicating that PKD1 was not activated by LIF treatment suggests that PKD1 is a component of a cardiac hypertrophy signal transduction mediated through the seven transmembrane-spanning heterotrimeric G protein-coupled receptor (GCPR) signaling pathway, and is deeply related to the cardiac hypertrophy onset mechanism through this pathway. Moreover, the mechanism by which EGF produces cardiac hypertrophy is based on receptor-type tyrosine kinase, which is not at all mediated through GPCR, and therefore this suggests that PKD1 mediates downstream pathway through EGF receptor, as well as GPCR signaling pathway.

### Experiment 4

Control of PKD1 Fully Activation (Phosphorylation)

The ability of PKD1 to phosphorylate Syntide-2 (APLARTLSVAGLPGKK), which is a selective substance for PKD1, can be used as an index to assess PKD1 activity (Valverde, A. M., et al., Proc. Natl. Acad. Sci. USA, 91, 1994, p. 8572-8576). Accordingly, the phosphorylation activity of PKD1 within cardiomyocytes with induced cardiac hypertrophy was evaluated based on the ability to phosphorylate Syntide-2.

Concretely, immunoprecipitation kinase assays using Syntide-2 as a substrate were conducted on the following NRC samples: NRC samples with cardiac hypertrophy induced by treating for 20 minutes respectively with \textcircled{1} 100 nM TPA, \textcircled{2} 100 μM NE, and \textcircled{3} 20 nM LIF, and NRC samples treated for 20 minutes respectively with \textcircled{4} 400 nM GF109203X (PKC inhibitor), \textcircled{5} 20 nM TPA+400 nM GF109203X, \textcircled{6} 100 μM NE+400 nM GF109203X, and \textcircled{7} 20 nM LIF+400 nM GF109203X. The phosphorylation activity of PKD1 present in the various NRC samples was measured. In addition, as a control, the phosphorylation activity of PKD1 present in NRC that was not treated with anything (untreated NRC) was described and measured in the same way.

(1) Immunoprecipitation Kinase Assay

NRC prepared by the same method as described in (1) of Experiment 1 was cultured over night (5% CO₂ concentration, 37° C.) in DMEM without blood serum (manufactured by Nacalai Tesque). To the cultured cells, \textcircled{1} 100 nM TPA, \textcircled{2} 100 μM NE (containing 2 μM propranol), \textcircled{3} 20 nM LIF, \textcircled{4} 400 nM GF109203X (PKC inhibitor), \textcircled{5} 20 nM TPA+400 nM GF109203X, \textcircled{6} 100 μM NE+400 nM GF109203X, and \textcircled{7} 20 nM LIF+400 nM GF109203X were added respectively, and treated for 20 minutes. The cells obtained were treated with Lysis buffer solution A, and incubated for 60 minutes on ice together with anti-fully active PKD1 polyclonal antibody. This was treated with protein G Sepharose 4B, and protein bound to the resin (Protein G Sepharose 4B bound PKD1-anti-PKD1 antibody complex) was recovered. The PKD1 immunoprecipitate was rinsed 1 time in Lysis buffer solution A, and further rinsed 2 times with Lysis buffer solution A not containing NaF.

The PKD1 was eluted from PKD1 immunoprecipitate obtained by incubating in Lysis buffer solution A not containing NaF together with 100 μL of 0.5 mg/mL immunizing peptide. The immunizing peptide is an antigen peptide used in order to immunize test animals to create anti-fully active PKD1 polyconal antibody. When incubating this peptide with protein G Sepharose 4B bound PKD1-anti-PKD1 antibody complex, the aforementioned antigen peptide and the anti-PKD1 antibody bond, and as a result, PKD1 can be freed and obtained.

The eluted PKD1 (10 μL) was incubated for 5 minutes at 30° C. together with assay mixed solution (15 μL Tris/MgCl₂ (100 mM Tris, 100 mM MgCl₂), 5 μL ATP (800 μM), 0.2 μL ³²γ-ATP, and 40 μg Syntide-2). Next, the PKD1 phosphorylation activity was measured by liquid scintillation counter (n=9).

(2) Results

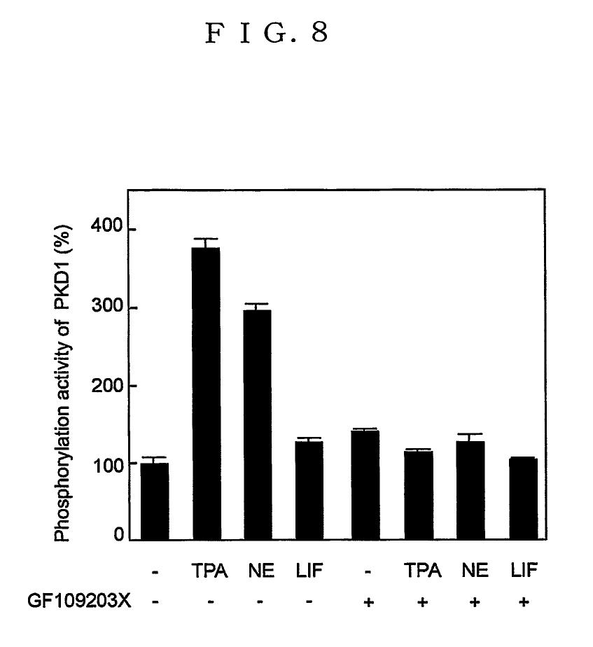

The results are indicated in FIG. 8. Indicated from the left in the bar graph are the levels of phosphorylation activity of PKD1 present in untreated NRC (negative control), and in NRC treated with \textcircled{1} 100 nM TPA, \textcircled{2} 100 μM NE (containing 2 μM propranol), \textcircled{3} 20 nM LIF, \textcircled{4} 400 nM GF109203X (PKC inhibitor), \textcircled{5} 100 nM TPA+400 nM GF109203X, \textcircled{6} 100 nM NE+400 nM GF109203X, and \textcircled{7} 20 nM LIF+400 nM GF109203X. In the diagram, the levels of phosphorylation activity of PKD1 present in the various types of NRC are indicated in the relative percentages (%) to when the measured level of the negative control (CPM, ³²P) is taken as 100%.

As indicated in FIG. 8, the PKD1 present in NRC treated by either TPA or NE both has notably higher phosphorylation activity than the PKD1 present in untreated NRC (negative control) or in NRC treated with LIF. Meanwhile, when treating the NRC simultaneously with these cardiac hypertrophy inducers (TPA, NE) and PKC inhibitor (GF109203X), the intrinsic PKD1 activity was suppressed to the same level as that of the negative control.

These facts demonstrate that phosphorylation of PKD1 in cardiomyocytes arises based on activation of PKC. Moreover, the intrinsic PKD1 activity of cardiomyocytes treated with LIF is hardly different from that of untreated NRC. These results, as indicated in aforementioned Experiment 3, support the fact that PKD1 is a component related to a cardiac hypertrophy signaling pathway mediated through GPCR induced by NE that is independent from the cardiac hypertrophy signaling pathway induced by LIF.

### Experiment 5

Subtype of PCK that Controls PKD1 Activity

Experiments 3 and 4 demonstrated that PKC activation is related to full activation of PKD1 produced in connection with cardiac hypertrophy. Here, we studied what subtypes are related to the full activation of PKD1, because it is known that there are multiple subtypes of PLK.

For this purpose, concretely, NRC transiently expressed kinase dead PKCs (PKCα, PKCβI, PKCδ, PKCε, PKCξ) was treated with a cardiac hypertrophy inducer (NE) in the same manner as in Experiment 4, and the PKD1 phosphorylation activity present in the NRC obtained was measured by immunoprecipitation kinase assay.

(1) Preparation of Cardiomyocytes that Express Kinase Dead PKCs

DNA coding for the kinase dead PKC (PKCα, PKCβI, PKCδ, PKCε, or PKCξ) was introduced into NRC prepared using a method similar to the method described in (1) of Experiment 1 in 10 cm-diameter plastic dishes, by using transfection reagent (Duo Fect: Q-biogen) according to the instruction manual, to obtain transfored NRC enable to express the aforementioned kinase dead PKC.

(2) Immunoprecipitation Kinase Assay

The aforementioned NRC that suppressed intrinsic PKCs activity by transient expression of various kinase dead PKCs (PKCα, PKCβI, PKCδ, PKCε, or PKCξ) was cultured over night in DMEM without blood serum (manufactured by Nacalai Tesque) (5% CO₂ concentration, 37° C.), added 100 μM of NE (containing 2 μM propranol), and then each of mixtures was processed for 20 minutes. The various NRC samples obtained were processed in the same manner as in Experiment 4 (1), and the PKD1 was collected. The PKD1 (10 μL) obtained was incubated at 30° C. for 5 minutes together with assay mixed solution in the same manner as in Experiment 4 (1), and the PKD1 phosphorylation activity was measured using a liquid scintillation counter (n=9). As a comparative experiment, phosphorylation activity was measured in the same way for PKD1 present in cardiomyocytes treated with NE using wild type NRC, and for PKD1 present in cardiomyocytes untreated with NE using wild type NRC, as a negative control.

(3) Results

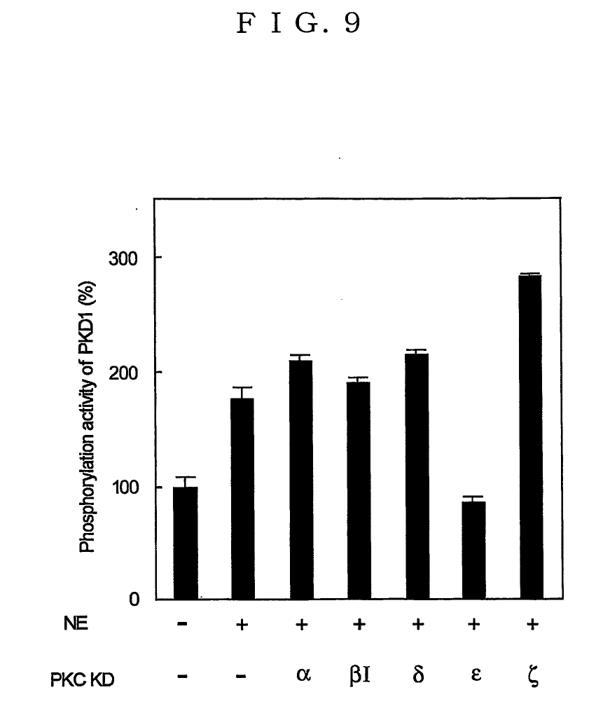

The results are indicated in FIG. 9. Indicated from the left in the bar graph are the levels of phosphorylation activity of PKD1 present in the following cardiomyocytes: \textcircled{1} untreated wild type NRC (negative control), \textcircled{2} wild type NRC+NE treatment, \textcircled{3} NRC-expressed kinase dead PKCα+NE treatment, \textcircled{4} NRC-expressed kinase dead PKCβI+NE treatment, \textcircled{5} NRC-expressed kinase dead PKCδ+NE treatment, \textcircled{6} NRC-expressed kinase dead PKCε+NE treatment, and \textcircled{7} NRC-expressed kinase dead PKCξ+NE treatment.

As indicated in FIG. 9, intrinsic PKD1 activity in the cells is the same level as PKD1 activity of the untreated wild type NRC (negative control) only when kinase dead PKCε is introduced into the NRC (specifically, only NRC with suppressed PKCε activity), and demonstrates that PKC1 was not fully activated (phosphorylated) even when inducing cardiac hypertrophy with NE. This fact indicates that, of the PKC subunits, PKCε is the activator positioned upstream of PKD1 in cardiac hypertrophy signaling pathway in cardiomyocytes.

### Experiment 6

Interaction of PKCε and PKD1

The experimental results above demonstrate that activation of PKCε and PKD1 are related to cardiac hypertrophy signal transduction mediated through GPCR, and that PKCε is an activator positioned upstream of PKD1. Therefore, the interaction between PKCε and PKD1 in cardiomyocytes was studied next. Specifically, we studied the interaction between PKCε and PKD1 in NRC with cardiac hypertrophy induced by NE (100 μM).

(1) Immunoprecipitation Assay

Primary NRC in culture prepared according to the method described in Experiment 1 (1) was treated with 100 μM NE (containing 2 μM propranol) in the same manner as in Experiment 3; the NE treated NRC in question was treated with Lysis buffer solution A; and 1 mL of cell extract (cell lysate) was obtained. Three hundred milliliters of the cell extract was incubated for 60 minutes on ice together with anti-PKD1/2 monoclonal antibody; and this was then treated with protein G Sepharose 4B, and protein bound to the resin in question (anti-PKD1 antibody immunoprecipitate sample) was recovered. As indicated in experiment 1, no PKD2 was present in the NRC. Using anti-PKDε monoclonal antibody made in the same manner, anti-PKDε antibody immunoprecipitate sample was collected. As a control, 1 mL cell extract (cell lysate) of untreated NRC was obtained in the same way, and anti-PKDε antibody immunoprecipitate sample and anti-PKD1 antibody immunoprecipitate sample were prepared.

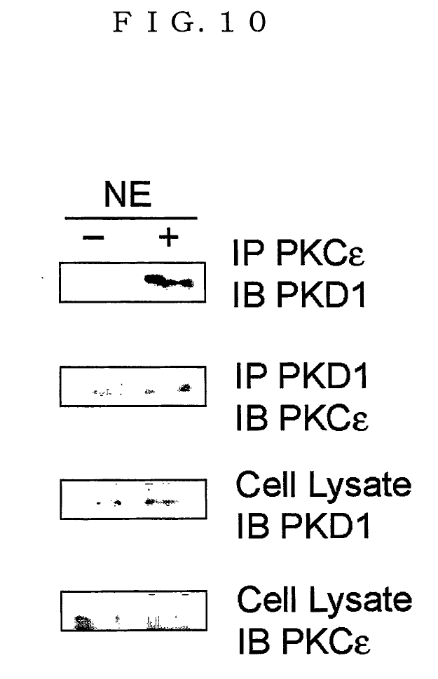

Next, Western blotting was conducted on the anti-PKCε antibody immunoprecipitate samples and the cell extract (cell lysate) obtained for the various NRC by using anti-PKD1/2 monoclonal antibody as the first antibody, and anti-mouse or anti-rabbit IgG bound with horseradish derived peroxidase (manufactured by Amersham Pharmacia) as the second antibody. The results relating to the anti-PKD antibody immunoprecipitate samples are indicated in the first (upper) column of FIG. 10, and the results relating to the cell lysate are indicated in the third column.

Western blotting was conducted on the anti-PKD1/2 antibody immunoprecipitate samples and cell lysate obtained from the various NRC using anti-PKCε monoclonal antibody as the first antibody and the same second antibody as above. The results relating to the anti-PKD1/2 antibody immunoprecipitate samples are indicated in the second column of FIG. 10, and the results relating to the cell lysate are indicated in the forth column of FIG. 10.

As indicated in the first and third columns, anti-PKD1/2 antibody immunoprecipitate samples (specifically, samples containing PKD1) and anti-PKDε antibody immunoprecipitate samples (specifically, samples containing PKCε) of NE-treated NRC reacted with anti-PKCε antibodies and anti-PKD1/2 antibodies, respectively. This fact demonstrates that complex of PKCε and PKD1 were formed in the NRC with induced cardiac hypertrophy. This was not observed in NRC not treated with NE, and therefore appears to indicate that PKCε activated in the process of generating cardiac hypertrophy (cardiac hypertrophy signaling process) reacts with PKD1 and forms a complex. In this way, it was confirmed that in the process of hypertrophy of cardiomyocytes, PKD1 interacts with PKCε, which was already known to be related to the inducement of cardiac hypertrophy, and this further suggests that PKD1 is one of the primary constitutive proteins of cardiac hypertrophy signals.

It has been confirmed that PKCε and PKD1 interact in vitro (Waldron, R. T., et al., J. Biol. Chem. 1999, 274, p. 9224-9230), but this was nothing more than simply observing the interaction of both by forcing the expression of PKCε and PKD1 in culture cells of an established cell line. The results in the aforementioned Experiment 6 are the first to establish that PKCε and PKD1 in cardiomyocytes with cardiac hypertrophy (treated with 100 μM NE) form a complex by mutual activation and interaction.

### Experiment 7

Inducement of Cardiac Hypertrophy by Fully Active PKD1

The above experimental results demonstrated that PKD1 was fully activated (phosphorylated) in cardiomyocytes in which cardiac hypertrophy had been caused. Here, we studied whether or not cardiac hypertrophy is induced by fully activating (phosphorylating) of PKD1 (whether or not fully active PKD1 induces cardiac hypertrophy).

(1) Concretely, a plasmid having DNA coding for GFP fused-constitutively active PKD1 (GFP-PKD1/CA), or as a control, DNA coding for GFP (GFP) was introduced into NRC by using the transfection reagent (Duo Fect: Q-biogen) according to the instruction manual, to obtain transformed NRC.

The cells obtained above (transformants) were incubated at 4° C. for 16 hours in blocking buffer solution containing antibodies (anti-sarcomeric-α-actinin monoclonal antibodies). This was rinsed 2 times in PBS, then was transferred to PBS containing with 0.3 v/v % Cy3-labelled anti-mouse IgG antibody (second antibody) (manufactured by Amersham Pharmacia), and was incubated for 1 hour at room temperature. The luminescent signals of the cells were observed by a confocal laser microscope LSM5 Pascal (manufactured by Carl Zeiss, Ltd.).

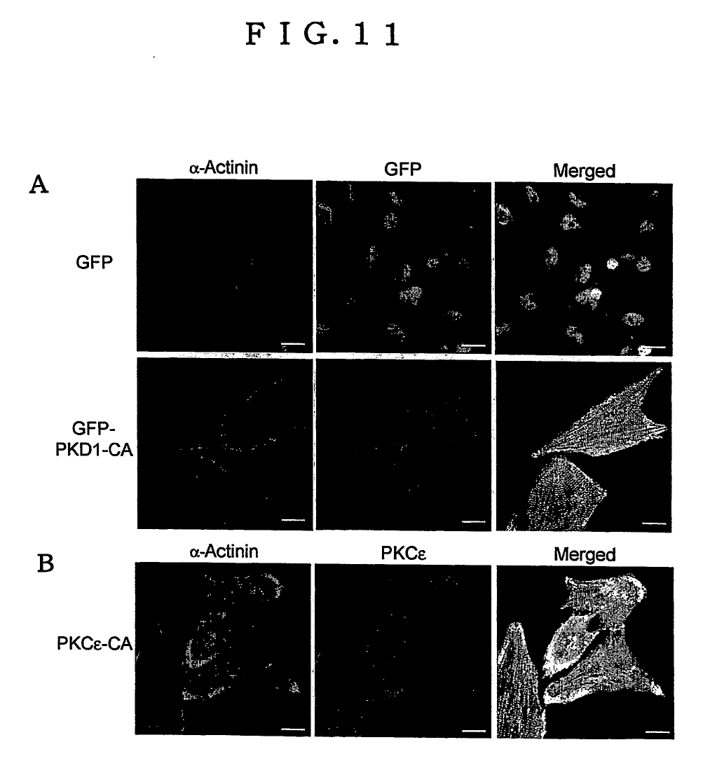

The results are indicated in FIG. 11A. In the NRC (middle lower panels) in which fully active PKD1 were introduced (GFP-PKD1 CA), the cell size were increased and sarcomere structures (Z-discs) were clearly formed compared to the control (upper panels), and this fact demonstrates that cardiac hypertrophy was introduced. Furthermore, lower panel in FIG. 11A indicates that fully active PKD1 spontaneouly localized at Z-discs, which suggests the possibility that the fully active PKD1 alone induces the formation of Z-discs

(2) a plasmid having DNA coding for constitutively active PKCε (PKCε/CA) by electroporation using the Amaxa electroporator with the Rat Cardiomyocyte-Neonatal Nucleofector kit (manufactured by Amaxa GmbH) according to the manufacture's instructions.

The cells obtained above (transformants) were processed by the same manner as described in (1), the luminescent signals of the cells were observed by a confocal laser microscope LSM5 Pascal (manufactured by Carl Zeiss, Ltd.).

The results are indicated in FIG. 11B. FIG. 11B indicates that active PKCε alone spontaneouly translocated at Z-discs.

### Experiment 8

Relationship Between Active PKC and the Inducement of Cardiac Hypertrophy

The identification the PKC isoforms (PKCε, PKCβI, and PKCδ; these are the main PKCs in the heart) that induce, cardiac hypertrophy was attempted, using various PKC expression plasmids.

Concretely, in a similar manner to Experiment 7, NRC was transformed by introduction of an expression vector having DNA coding for constitutively active PKC mutant (CA-PKCε, CA-PKCβI, or CA-PKCδ) or dominant negative PKC mutant (K440M PKCε). These transformed cells were stained with anti-sarcomeric-α-actinin antibody and the second antibody, and the presence of hypertrophy was observed using a confocal laser microscope (LSM5 Pascal: Carl Zeiss). The NRC, in which dominant negative PKC mutant (K440M PKCε) was expressed, was treated with the cardiac hypertrophy inducer, NE or LIF, and was cultured for 38 hours, and then immunostaining was conducted.

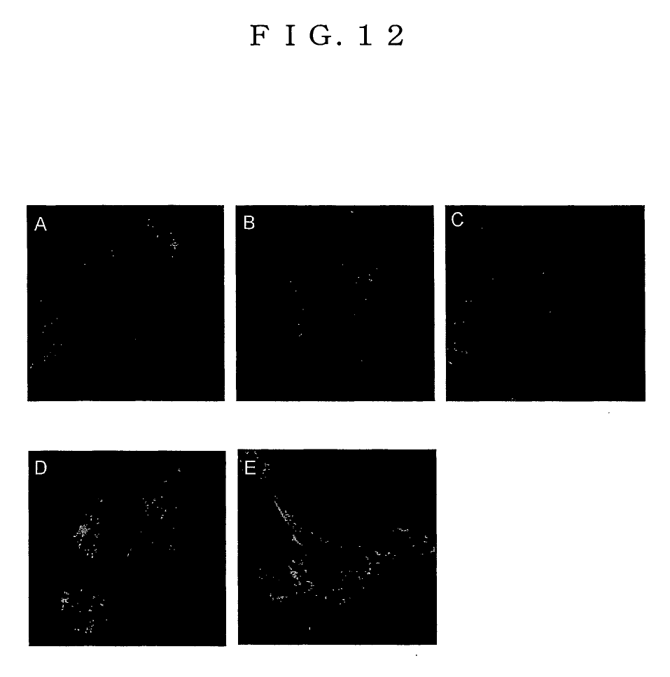

The results are indicated in FIG. 12.

These are images in which anti-sarcomeric-α-actinin antibody and the second antibody were allowed to react with NRCs and the α-actinin was visualized. Panel A indicates image of NRC in which constitutively active PKCε (CA-PKCε) was introduced; panel B indicates image of NRC in which constitutively active PKCβI (CA-PKCβI) was introduced; panel C indicates image of NRC in which constitutively active PKCδ (CA-PKCδ) was introduced; panel D indicates image of NRC in which dominant negative PKCε (K440R PKCε) was introduced, and the cells were treated with 100 μM of NE (induced cardiac hypertrophy treatment); and panel E indicates image of NRC in which dominant negative PKCε (K440R PKCε) was introduced, and the cells were treated with 20 nM of LIF (induced cardiac hypertrophy treatment).

As shown in FIG. 12, notable onset of cardiac hypertrophy was observed in NRC in which constitutively active PKCε was expressed (panel A). Meanwhile, NRC in which dominant negative PKCε was expressed did not generate cardiac hypertrophy even when treating with the cardiac hypertrophy inducer NE (panel D). These facts demonstrate that among the isoforms of PKC, PKCε participates in cardiac hypertrophy signal transduction.

### Experiment 9

Activate PKCε and Fully Activate PKD that Induce Cardiac Hypertrophy

The levels of expression of arterial natriuretic factors (ANF) (hypertrophy markers: Tsuchimochi H., et al., Lancet, 1987 Aug. 8. 2 (8554) p. 336-7) were compared for various types of transformed NRC. Concretely, in a similar manner to Experiment 7, various types of transformants were created by introducing into NRC plasmids having DNA coding for constitutively active PKCs (CA-PKCε, CA-PKCβI, CA-PKCδ), kinase dead PKCε (KD-PKCε), constitutively active PKD1 (CA-PKD1), dominant negative PKD1 (DN-PKD1), or GFP. Using a similar method to (2) of Experiment 7, immunostaining of the transformed cells obtained and the cells additionally treated with a cardiac hypertrophy inducer, which are prepared by treating the transformed cells with the cardiac hypertrophy inducer (NE or LIF), was conducted using anti-ANF antibody as the first antibody, and 0.3 v/v % Cy2-labelled anti-rabbit IgG antibody (manufactured by Amersham Pharmacia) as the second antibody. Three dishes, in which NRC was grown to the same number of cells (10⁵), were used for each type,of transformed cell; images of transformed cells were observed using confocal laser microscope (LSM5 Pascal: Carl Zeiss) (3 images per dish); and the intensity of fluorescence of the image data of the various cells (specifically, ANF expression level in the various cells) were measured using image analysis software Scion Image (manufactured by Scion Corporation).

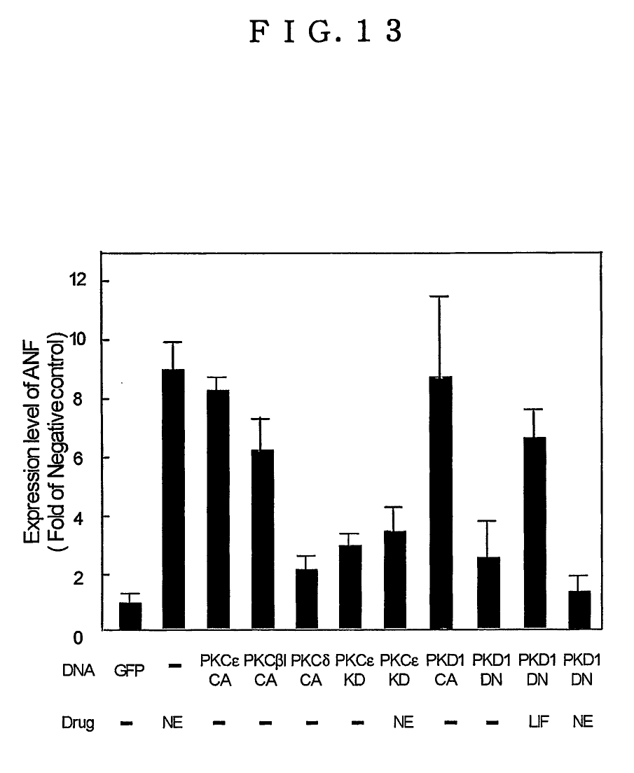

The results are indicated in FIG. 13. Indicated from the left in the bar graph are the expression levels of ANF in the following NRC types: GFP expression+untreated (negative control), wild type+NE treatment (positive control), CA-PKCε expression+untreated, CA-PKCβI expression+untreated, CA-PKCδ expression+untreated, KD-PKCε expression+untreated, KD-PKCε expression+NE treated, CA-PKD1 expression+untreated, DN-PKD1 expression+untreated, DN-PKD1 expression+LIF treatment, and DN-PKD1 expression+NE treatment. These results confirmed that hypertrophy is generated in cardiomyocytes in which PKD1 is fully activated by introducing constitutively active PKD1 (CA-PKD1), or in which PKCε is activated by introducing constitutively active PKCε (CA-PKCε). These results agree with the results of the aforementioned Experiments 7 and 8, and support the fact that PKCε activation and PKD full activation are a central, necessary and indispensable cascade processes in cardiac hypertrophy signaling mediated through GPCR.

<Discussion>

The above experiments demonstrate that cardiac hypertrophy is caused by activation of PKD1 directly induced by PKCε, in the cardiomyocytes of mammals.

Concretely, cardiac hypertrophy was induced in cardiomyocytes by full activation (phosphorylation) of PKD1 or activation of PKCε (Experiments 7 to 9). Meanwhile, cardiac hypertrophy was not caused in cardiomyocytes into which dominant negative mutants of PKD1 and PKCε have been introduced, even if treatment with the cardiac hypertrophy inducer (AngII or NE) was done (Experiments 7 to 9). From these facts, it appears that activation of PKCε and full activation (phosphorylation) of PKD1 are central cascade processes of cardiac hypertrophy signaling induced by AngII and NE in cardiomyocytes (cardiac hypertrophy signaling mediated through GPCR). Moreover, because PKCε activation is necessary in full activation of PKD1 in cardiomyocytes and in inducing cardiac hypertrophy thereby (Experiments 4 and 5), and because complex formations of PKCε and PKD1 were observed in cardiomyocytes with cardiac hypertrophy (Experiment 6), it appears that PKCe is an activator positioned upstream of PKD1 in cardiac hypertrophy signaling mediated through GPCR in the cardiomyocytes of mammals, and that cardiac hypertrophy signal transduction is carried out by PKD1 being activated in conjunction with the activation of PKCε, and by forming a complex of the two.

In the aforementioned experiments, the focus was on the cardiac hypertrophy signaling pathway mediated through GPCR induced by NE and AgnII, but because full activation of PKD1 and movement thereof to sarcomere Z-discs was also observed in the same way in cardiomyocytes in which EGF was allowed to act, it appears that PKD1 is positioned downstream of signaling through EGF receptors, and it appears that the cascade mediated through PKD1 is present in the cardiac hypertrophy system mediated by EGF (cardiac hypertrophy system based on receptor-type tyrosine kinase, which is not at all mediated through GPCR), as well as in the cardiac hypertrophy signaling pathway mediated through GPCR.

### Experiment 10

Action to Suppress Cardiac Hypertrophy Based on ENH2

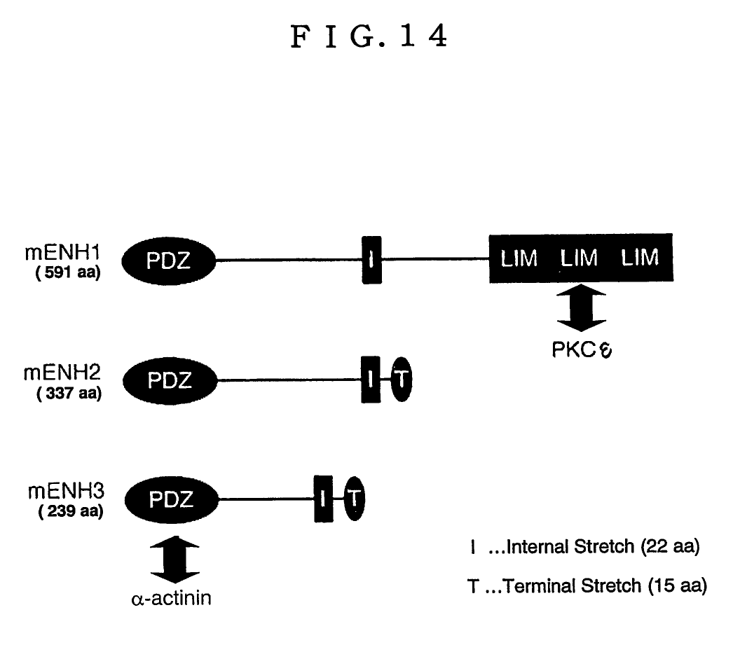

ENH1 (enigma homologue protein 1) is a protein of approximately 60 kDa present specifically in heart and skeletal muscles that was isolated and identified from a rat brain-derived cDNA library by yeast two-hybrid screening using the control region of PKCβI as bait. ENH1 has a PDZ domain on the N-terminal side, and 3 LIM domains on the C-terminal side. Moreover, multiple splice variants ENH2 (human, mouse, rat) and ENH3 (mouse, rat) having LIM domains deleted, alternatively having homologous sequences, called the “T-stretch”, were discovered (refer to FIG. 14).

When using a phorbol ester (for example, TPA, etc.), which is a PKC activator, to induce a cardiac hypertrophy state in NRC, ENH1 bonds with α-actinin mediated through the PDZ domain, and is translocated to sarcomere Z-discs. More recently there has been a report that ENH1 is a scaffold protein that recruits PKCβ to Z-discs mediated through the LIM domain (Nakagawa N., et al., Biochem. Biophys. Res. Commun., 2000, 272(2), p. 505-512).

After the aforementioned report, the present inventors discovered that PKD1, which is phosphorylated and fully activated directly by PKCε as demonstrated by the previously described experimental results, interacts with ENH1. And the present inventors confirmed the fact that the regions of this interaction are the catalytic region positioned in the C-terminal region of PKD1 and the LIM domain of ENH1. The inventors discovered that fully active PKD1 and ENH1 move to sarcomere Z-discs and are localized there in cardiomyocytes with cardiac hypertrophy induced by cardiac hypertrophy inducers mediated through GCPR, such as NE, AngII, etc. On the other hand, cardiac hypertrophy inducers mediated through gp130 receptors such as LIF, etc. do not activate PKD1 nor cause movement to the Z-discs. These facts demonstrate that PKD1 is activated by signaling mediated through GPCR, and moves together with ENH1 to cardiomyocyte sarcomere Z-discs and becomes localized there. The present inventors further discovered that activated PKD1, PKCε, and ENH1 interact in cardiac hypertrophy cardiomyocytes to form a complex, and localize on cardiomyocyte sarcomere Z-discs (none of this has been published).

(1) Experiment

NRC prepared by the same method as described in (1) of Experiment 1 was moved to a glass culture plate (35 mm diameter) coated with poly-L-lysine, and was cultured over night (5% CO₂ concentration, 37° C.) in DMEM without blood serum (manufactured by Nacalai Tesque). Following the product manuals of transfection reagent (Duo Fect: Q-biogen), plasmids introduced DNA coding for FLAG fused-ENH1 or FLAG fused-ENH2 were suspended with the transfection reagent (Duo Fect: Q-biogen), each of the prepared suspensions was added to NRC, thereby NRC was transformed. The cells were cultured in DMEM medium containing 10% FBS. One day after transforming, the medium was replaced with DMEM medium without blood serum, each of the transformants (FLAG fused-ENH1 expressed NRC, FLAG fused-ENH2 expressed NRC) was divided in two samples, and one of each was treated with 20 nM TPA, preparing TPA-treated NRC sample and untreated NRC sample.

After approximately 18 hours, the TPA-treated NRC sample and untreated NRC sample of each of the transformants (FLAG fused-ENH1 expressed NRC, FLAG fused-ENH2 expressed NRC) were double stained with anti-ANF polyclonal antibody (T4015, manufactured by Peninsula Laboratories Inc.) and anti-FLAG monoclonal antibodies (manufactured by Sigma) using the same method as in Experiment 3, and the cell luminescent signals were observed by confocal laser microscope.

(2) Results

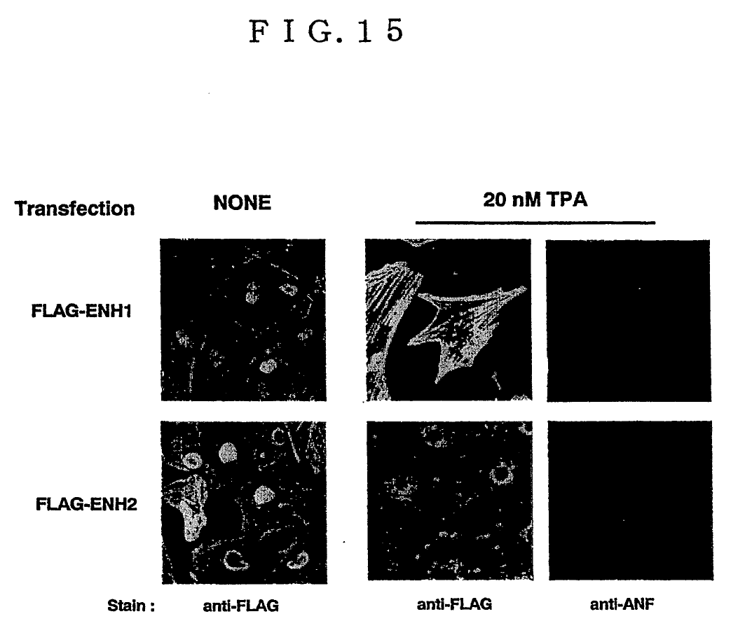

The results are indicated in FIG. 15. As indicated in the figure, when NRC-expressed ENH1 was treated with TPA, sarcomeres were formed, ANF expression increased, and cardiac hypertrophy was observed, but when NRC-expressed ENH2 was treated with TPA, no sarcomeres were formed and the amount of ANF expressed decreased. These facts suggest that a deletion mutant “ENH2” which does not have LIM domains of ENH1 is an “intrinsic anti-cardiac hypertrophy antagonist” that suppresses and blocks the cardiac hypertrophy signal cascade, because the ENH2 competitvely inhibits the link of PKC to sarcomere Z-discs caused by ENH1. These facts also imply that a deletion mutant “ENH3” derived from mouse or rat, which does not have LIM domains as the ENH2, acts as an “intrinsic anti-cardiac hypertrophy antagonist” that competitvely inhibits the link of PKC to sarcomere Z-discs caused by ENH1, and suppresses and blocks the cardiac hypertrophy signal cascade.

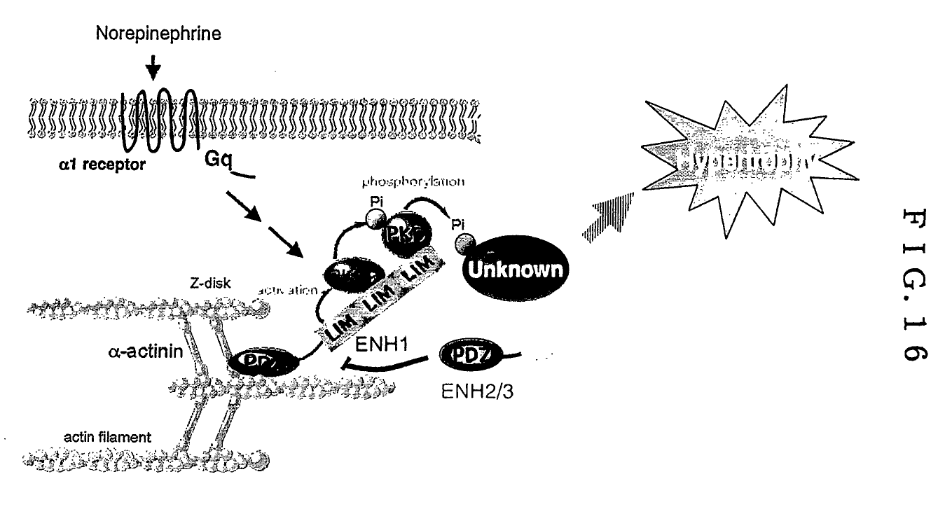

A conceptual diagram of a cardiac hypertrophy signal control model is indicated in FIG. 16. The above results suggest as follows: in the cardiac hypertrophy signal transduction mediated through GPCR, when cardiac hypertrophy is induced in cardiomyocytes, ENH1 holds a cardiac hypertrophy signaling factor on the sarcomere Z-discs, PKD1 is activated by PKCε while held on said Z-discs, and phosphorylation signals are transmitted further downstream in the cardiac hypertrophy signaling pathway; and that ENH2, which is an ENH1 mutant with deleted LIM domains, suppresses the cardiac hypertrophy signaling mediated through ENH1, and acts as an intrinsic antagonist to regulate and suppress cardiac hypertrophy. In addition, the above results also suggest that it is possible to suppress cardiac hypertrophy, by suppressing the expression of ENH1 or the functional activity thereof in cardiomyocytes to block cardiac hypertrophy signal cascade.

### INDUSTRIAL APPLICABILITY

A composition for suppressing cardiac hypertrophy of the present invention, which comprises as an active ingredient a substance that suppresses functional expression of PKD1 in cardiomyocytes, can prevent or suppress inducement of cardiac hypertrophy by suppressing the functional expression in cardiomyocytes of PKD1, which plays a central role in the cardiac hypertrophy signal cascade. A composition for suppressing cardiac hypertrophy of the present invention, which comprises as an active ingredient a nucleic acid (DNA) that has a base sequence coding for ENH2, can prevent or suppress inducement of cardiac hypertrophy by inhibiting the function of ENH1, which holds cardiac hypertrophy signaling factors during hypertrophy of cardiomyocytes. For this reason, a composition for suppressing cardiac hypertrophy of the present invention can suppress the onset and development of cardiac hypertrophy by suppressing the inducement of cardiac hypertrophy, and can be effectively used as a pharmaceutical composition to prevent or remedy diseases caused by cardiac hypertrophy, specifically, heart failure, ischemic heart disease, or arrhythmia.

It was newly discovered that PKD1 is deeply related as a major component in the inducement of cardiac hypertrophy mediated through cardiac hypertrophy signaling pathway, and that ENH2 is an intrinsic antagonist of ENH1 related to the inducement of cardiac hypertrophy, and the present invention provides these findings. Consequently, by using these findings related to the cardiac hypertrophy onset mechanism that the present invention provides, it, is possible to construct a method for screening active ingredients to suppress the onset of cardiac hypertrophy or active ingredients to prevent or remedy heart diseases caused by cardiac hypertrophy. By using the cardiac hypertrophy suppression substances obtained by the screening method in question, it is possible to prepare and provide effective compositions to prevent or remedy heart diseases such as heart failure, ischemic heart disease, or arrhythmia.

Moreover, by using the aforementioned findings relating to the cardiac hypertrophy onset mechanism, it is possible to create and provide non-human animal models of cardiac hypertrophy having specific characteristic that resemble a human cardiac hypertrophy disease. The non-human animal models of cardiac hypertrophy can be effectively used to conduct histological research on cardiac hypertrophy, to clarify the mechanisms of the development into cardiac hypertrophy, and to screen active ingredients for the development of cardiac hypertrophy suppressants and of preventative and remedial agents for heart disease.

1. A pharmaceutical composition for suppressing cardiac hypertrophy, which comprises a substance that suppresses functional expression of protein kinase D1 in cardiomyocytes as an active ingredient.

2. The pharmaceutical composition for suppressing cardiac hypertrophy according to claim 1 wherein the active ingredient is a substance that has an action to suppress the expression of protein kinase D1 genes, or the activation or phosphorylation of the product thereof, in cardiomyocytes.

3. The pharmaceutical composition for suppressing cardiac hypertrophy according to claim 1, wherein the active ingredient is nucleic acid having a base sequence that codes for dominant negative protein kinase D1 which has been controlled to be able to express in cardiomyocytes.

4. The pharmaceutical composition for suppressing cardiac hypertrophy according to claim 1, wherein the active ingredient is an antisense molecule, ribozyme, or RNAi effector that suppresses the expression of protein kinase D1 genes in cardiomyocytes.

5. A pharmaceutical composition for suppressing cardiac hypertrophy, which comprises nucleic acid having a base sequence that codes for ENH2 as an active ingredient.

6. The pharmaceutical composition for suppressing cardiac hypertrophy according to claim 1 or 5, wherein cardiac hypertrophy is induced by hypertrophy signal transmission through seven transmembrane-spanning heterotrimeric G protein-coupled receptor or epidermal growth factor receptor.

7. A method to suppress cardiac hypertrophy or prevent onset of cardiac hypertrophy in a patient with cardiac hypertrophy or pre-conditions thereof, which comprises administering to said patient an effective amount of a substance that suppresses functional expression of protein kinase D1 in cardiomyocytes.

8. The method according to claim 7, wherein said substance is a substance that has an action to suppress the expression of protein kinase D1 genes, or the activation or phosphorylation of the product thereof, in cardiomyocytes.

9. The method according to claim 7, wherein said substance is nucleic acid having a base sequence that codes for dominant negative protein kinase D1 which has been controlled to be able to express in cardiomyocytes.

10. The method according to claim 7, wherein said substance is an antisense molecule, ribozyme, or RNAi effector that suppresses the expression of protein kinase D1 genes in cardiomyocytes.

11. A method to suppress cardiac hypertrophy or prevent onset of cardiac hypertrophy in a patient with cardiac hypertrophy or pre-conditions thereof, which comprises administering to said patient an effective amount of nucleic acid having a base sequence that codes for ENH2.

12. The method according to claim 7 or 11, wherein cardiac hypertrophy is induced by cardiac hypertrophy signal transduction through seven transmembrane-spanning heterotrimeric G protein-coupled receptor or epidermal growth factor receptor.

13. A pharmaceutical composition for preventing or remedying the onset of heart disease caused by cardiac hypertrophy, which comprises a substance that suppresses functional expression of protein kinase D1 in cardiomyocytes as an active ingredient.

14. The pharmaceutical composition according to claim 13, wherein the active ingredient is a substance that has an action to suppress the expression of protein kinase D1 genes, or the activation or phosphorylation of the product thereof, in cardiomyocytes.

15. The pharmaceutical composition according to claim 13, wherein the active ingredient is nucleic acid having a base sequence that codes for dominant negative protein kinase D1 which has been controlled to be able to express in cardiomyocytes.

16. The pharmaceutical composition according to claim 13, wherein the active ingredient is an antisense molecule, ribozyme, or RNAi effector that suppresses the expression of protein kinase D1 genes in cardiomyocytes.

17. A pharmaceutical composition, which comprises as an active ingredient nucleic acid having a base sequence that codes for ENH2.

18. The pharmaceutical composition according to claim 13 or 17, wherein cardiac hypertrophy is induced by hypertrophy signal transduction through seven transmembrane-spanning heterotrimeric G protein-coupled receptor or epidermal growth factor receptor.

19. A method to prevent or remedy onset of diseases caused by cardiac hypertrophy in a patient with cardiac hypertrophy or the preconditions thereof, which comprises administering to said patient an effective amount of a substance that suppresses functional expression of protein kinase D1 in cardiomyocytes.

20. The method according to claim 19, wherein said substance has an action to suppress the expression of protein kinase D1 genes, or the activation or phosphorylation of the product thereof in cardiomyocytes.

21. The method according to claim 19, wherein said substance is nucleic acid having a base sequence that codes for dominant negative protein kinase D1 which has been controlled to be able to express.

22. The method according to claim 19, wherein said substance is an antisense molecule, ribozyme, or RNAi effector that suppresses the expression of protein kinase D1 genes in cardiomyocytes.

23. A method to prevent or remedy onset of diseases caused by cardiac hypertrophy in a patient with cardiac hypertrophy or the preconditions thereof, which comprises administering to said patient an effective amount of nucleic acid having a base sequence that codes for ENH2.

24. The method according to claim 19 or 23, wherein cardiac hypertrophy is induced by hypertrophy signal transduction through seven transmembrane-spanning heterotrimeric G protein-coupled receptor or epidermal growth factor receptor.

25. A transgenic non-human animal, which a constitutively active protein kinase D1 is transiently expressed in cardiomyocytes.

26. The transgenic non-human animal according to claim 25, which is an animal model of cardiac hypertrophy or disease caused thereby.

27. A transgenic non-human animal, which a dominant negative protein kinase D1 is transiently expressed in cardiomyocytes.

28. A method for screening an active ingredient of cardiac hypertrophy suppressants, comprising the steps of:
(a) bringing a test substance into contact with cells that can express protein kinase D1;
(b) measuring the levels of expression of protein kinase D1 in said cells, and comparing with the levels of expression of protein kinase D1 in contrast cells that are not brought into contact with said test substance; and
(c) based on the comparative results of step (b) above, selecting as an active ingredient of cardiac hypertrophy suppressants the test substance which, when brought into contact with the cells, lower the level of expression of protein kinase D1, as compared to contrast cells.

29. A method for screening an active ingredient of cardiac hypertrophy suppressants, comprising the steps of:
(a) bringing a protein kinase D1 activator and a test substance into contact with cells that can express protein kinase D1;
(b) measuring the protein kinase D1 activity in said cells and comparing with the corresponding activity in contrast cells that are not brought into contact with the test substance; and
(c) based on the comparative results of step (b) above, selecting as an active ingredient of cardiac hypertrophy suppressants the test substance which, when brought into contact with the cells, lower the activity of protein kinase D1, as compared to contrast cells.

30. A method for screening an active ingredient of cardiac hypertrophy suppressants, comprising the steps of:
(a) bringing a test substance and a cardiac hypertrophy inducer that stimulate seven transmembrane-spanning heterotrimeric G protein-coupled receptor or epidermal growth factor receptor into contact with cardiomyocytes;
(b) measuring protein kinase D1 activity, localization of phosphorylated protein kinase D1 in sarcomeres of α-actinin, or the intermolecular distance of protein kinase Cε and protein kinase D1 in said cardiomyocytes, and comparing with the corresponding activity, localization or intermolecular distance in contrast cardiomyocytes that were brought into contact with hypercardia inducer only; and
(c) based on the comparative results of step (b) above, selecting as an active ingredient of cardiac hypertrophy suppressants the test substance which, when brought into contact with the cardiomyocytes, lower protein kinase D1 activity, lower localization of phosphorylated protein kinase D1 in α-actinin sarcomeres, or increase the intermolecular distance of protein kinase Cε and protein kinase D1, compared to the contrast cardiomyocytes.

31. A method for screening an active ingredient of cardiac hypertrophy suppressants, comprising the steps of:
(a) bringing a test substance into contact with cardiomyocytes that can express constitutively active protein kinase Cε or constitutively active protein kinase D1;
(b) measuring protein kinase D1 activity, localization of phosphorylated protein kinase D1 in sarcomeres of α-actinin, or the intermolecular distance of protein kinase Cε and protein kinase D1 in said cardiomyocytes, and comparing with the corresponding activity, localization or intermolecular distance in corresponding contrast cardiomyocytes that were not brought into contact with the test substance; and
(c) based on the comparative results of step (b) above, selecting as an active ingredient of cardiac hypertrophy suppressants the test substances which, when brought into contact with the cardiomyocytes, lower protein kinase D1 activity, lower localization of phosphorylated protein kinase D1 in α-actinin sarcomeres, or increase the intermolecular distance of protein kinase Cε and protein kinase D1, compared to the contrast cardiomyocytes.

32. A method for screening an active ingredient of cardiac hypertrophy suppressants, comprising the steps of:
(a) administering a test substance to the transgenic non-human animal according to claim 25;
(b) measuring the degree of cardiac hypertrophy of said non-human animal, and comparing with the degree of cardiac hypertrophy in contrast transgenic non-human animals that were not administered the test substance; and
(c) based on the comparative results of step (b) above, selecting as an active ingredient of cardiac hypertrophy suppressants the test substance that reduce or suppress cardiac hypertrophy.

33. The screening method according to any of claims 28 to 32, that is a method for obtaining active ingredients for pharmaceutical compositions to prevent or remedy heart disease caused by cardiac hypertrophy.
